(19) United States
(12) Patent Application Publication
(10) Pub. No.: US 2019/0105343 A1
(43) Pub. Date: Apr. 11, 2019

(54) Treatment of Skin Conditions and Diseases Associated with Microbial Biofilms

(71) Applicant: Bioclenz PH LLC, Charlestown (KN)

(72) Inventor: Keith BENSON, Reston, VA (US)

(21) Appl. No.: 16/087,449

(22) PCT Filed: Mar. 23, 2017
(86) PCT No.: PCT/US2017/023879

§ 371 Date: Sep. 21, 2018

Related U.S. Application Data
(60) Provisional application No. 62/312,515, filed on Mar. 24, 2016.
(60) Provisional application No. 62/312,518, filed on Mar. 24, 2016.
(60) Provisional application No. 62/312,524, filed on Mar. 24, 2016.

(51) Int. Cl. A61K 33/00; A61K 9/00; A61K 9/08; A61K 31/23; A61K 9/107; A61K 47/12; A61K 9/06; A61K 47/36; A61P 17/00; A61K 47/22; A61K 47/14; A61P 31/04

(52) U.S. Cl. CPC A61K 33/00; A61K 9/0014; A61K 9/08; A61K 31/23; A61K 9/107; A61P 31/04; A61K 9/06; A61K 47/36; A61P 17/00; A61K 47/22; A61K 47/14; A61K 47/12

(57) ABSTRACT

Compositions and methods for treatment, control, or prevention of skin conditions or skin diseases associated with microbial biofilms are provided. A topical formulation comprises a surfactant, pharmaceutically acceptable carrier, biocide, and weak acid and is effective to disrupt both the microbial biofilm defenses as well as the microbes within. The topical formulation is also provided as a multi-component system suitable for the application to human or animal skin or mucosa. Methods of administering the topical formulations to an individual having, or at risk for having, a skin disease or skin condition associated with a microbial biofilm are also disclosed.

Benefit is claimed of U.S. Provisional Application Nos. 62/312,515, filed Mar. 24, 2016, 62/312,518, filed Mar. 24, 2016, and 62/312,524, filed Mar. 24, 2016, the entire contents of each of which are incorporated by reference herein.

### FIELD

The field of the invention relates generally to antimicrobial agents and methods of their use. In particular, the invention provides methods and compositions to treat microbe-associated skin conditions by disrupting microbial biofilms to allow and enhance access of antimicrobial agents to the microbes contained therein.

### BACKGROUND

Biofilms are matrix-enclosed accumulations of microorganisms such as bacteria (with their associated bacteriophages), fungi, protozoa and viruses. While biofilms are rarely composed of a single cell type, there are common circumstances where a particular cellular type predominates. The non-cellular components are diverse and may include carbohydrates; both simple and complex; proteins, including polypeptides; lipids; and lipid complexes of sugars and proteins (lipopolysaccharides and lipoproteins).

Bacterial biofilms are comprised of an extracellular matrix that is produced by bacteria once they attach to a surface, which helps to protect the microbes from immune cells and antimicrobial agents. Since efficacy of antimicrobial agents (e.g., antibiotics, antiseptics, disinfectants, and antiviral compounds) is compromised by the extracellular biofilm matrix, strategies to disrupt the biofilm and expose microorganisms within can be helpful in increasing the activity level of antimicrobial agents and thus reducing the concentration of such agents needed to make an effective composition.

The architecture of biofilms is not simply an aggregation. Rather, biofilms are distinct communities that acquire new features and functions beyond those of their individual members. Because of the properties provided by microorganisms in a biofilm, microbes in biofilms are typically less susceptible to antibiotics, antimicrobials, biocides, and antiviral agents. In some cases, bacteria in a biofilm can be up to 4,000 times more resistant (i.e., less susceptible) than the same organism in a planktonic state.

Recent research has revealed that biofilm defenses are associated with many common skin diseases and conditions, such as eczema (atopic dermatitis), acne, warts, fungal diseases, and papilloma. The protective biofilms act as chemical and physical defenses to the microbes. This explains at least in part why many of these conditions do not respond, or respond only temporarily, to the most commonly prescribed treatments.

Eczema, also known as atopic dermatitis (AD), involves inflammation of the skin. The condition may cause significant discomfort in humans and other mammals and is characterized by scaled or crusty patches of skin, often accompanied by redness, blistering, itching, blemishes or skin lesions. Further, blemishes and lesions are often accompanied by inflammation of the skin glands and pilosebaceous follicles as well as microbial (especially bacterial) infection.

Recent population surveys suggest that the prevalence of AD is approximately 17% to 18%. The U.S. national estimated cost of treatment for this condition is as high as $3.8 billion per year. Eczema includes a wide variety of conditions. Some types of eczema include, for example, atopic eczema, contact eczema, seborrheic eczema, nummular eczema, neurodermatitis, stasis dermatitis, or dyshidrotic eczema. Biofilm formation by AD-associated staphylococci almost certainly plays a major role in the occlusion of sweat ducts and leads to inflammation and pruritus.

While topical steroidal or non-steroidal immuno-suppressive agents remain the primary treatment for atopic dermatitis and other skin conditions, they do not address the etiology of the disease, and some individuals do not respond to prescriptive medicines.

In addition to AD, microorganisms, especially Propionibacterium acnes, also are strongly implicated in the pathogenesis of acne and acne-associated skin orders. The microorganisms are thought to release microbial mediators of inflammation into the dermis or to trigger the release of cytokines from ductal keratinocytes. For example, acne vulgaris is an inflammatory dermatological disorder that occurs frequently in adolescence and, with some regularity, in older adults. The condition can include skin lesions ranging from the comedo in a pilosepaceous follicle, to more severe symptoms such as pustules, papules, cysts, and nodules. The condition can be uncomfortable and embarrassing and can result in scarring and facial disfigurement. The pathology is believed to involve a number of factors, including biofilm formation and defenses. See Nusbaum et al., Biofilms in Dermatology, Skin Therapy Lett. (2012 July; 17(7):1-5). Evidence is predominantly derived from the ability of P. acnes to form biofilms both in vitro and on implanted medical devices. The observed clinical trend towards decreased efficacy of topical antibiotics may be explained in part by the presence of biofilms, as biofilm-associated P. acnes exhibits increased resistance to commonly used anti-acne agents.

Psoriasis is another skin disorder that may be associated with microbial biofilms. Psoriasis is a chronic, widespread skin disorder afflicting millions of humans and even domesticated animals. The disorder is characterized by recurrent, elevated red lesions, plaques and, more rarely, pustules on the skin. The plaques are the results of an excessively rapid growth and shedding of epidermal (skin) cells. While the cause of psoriasis is unknown, it has been correlated with the presence of biofilms.

Additionally, many adverse skin conditions are associated with inflammation. Attacking, dissolving or otherwise weakening the bacterial biofilm matrix, interrupting the quorum mechanisms maintaining the bacterial community, as well as up-regulating local host innate immunity could cure what would otherwise become incurable chronic infection or chronic biofilm-associated inflammatory disease. Penetration or dispersion of the bacterial biofilm “armor” is critical in fighting biofilm-induced chronic inflammation.

Many other microbes and viruses, such as papilloma, are associated with biofilm and skin conditions, as described in Biofilm-based Healthcare-associated Infections, Volume 1 (Gianfranco Donelli ed., 2015), and Biofilm-based Healthcare-associated Infections, Volume 2 (Gianfranco Donelli ed., 2015).

From a microbiological perspective, the primary function of normal, intact human, and animal skin is to control microbial populations that live on the skin surface and to prevent underlying tissue from becoming colonized and invaded by potential pathogens. Exposure of subcutaneous tissue (i.e., a wound) provides a moist, warm, and nutritious environment that is conducive to microbial colonization and proliferation. Since wound colonization is mostly polymicrobial, involving numerous microorganisms that are potentially pathogenic, any wound, including a surgical wound, is at some risk of becoming infected.

Wounds are often colonized by a variety of microorganisms, some of which may cause infection. It is increasingly recognized that microbial populations living within a biofilm environment contribute to delayed wound healing and infection. Over the last few years, some have linked biofilm to chronic wounds. Microscopic evaluation of chronic wounds showed well organized biofilm with extracellular polymeric substance adhered around colony bacteria in at least 60% of the chronic wounds. Recent studies have identified microbial biofilms as potential causes for why some chronic wounds do not heal. See Singh and Barbul, Wound Rep Reg. 16: 1 (2008). In addition, James et al., Wound Rep Reg. 16: 37-44 (2008), has recently demonstrated biofilms in over 60% of bacterial infections associated with chronic wounds such as diabetic foot ulcers, venous leg ulcers, and pressure ulcers.

Most wound infections are caused by Staphylococcus aureus (20%), Staphylococcus epidermidis (14%), Enterococci spp. (12%), Escherichia coli (8%), Pseudomonas aeruginosa (8%), Enterobacter spp. (7%), Proteus spp. (3%), Klebsiella pneumoniae (3%), Streptococci (3%), and Candida albicans (3%), and overuse of antibiotics and the associated increase in bacterial resistance is impacting the efficacy of antibiotics in the treatment or prevention of wound infection. Effective alternatives to antibiotics are thus desirable.

The chronic wound infections are typically persistent infections that develop slowly, seem to be rarely resolved by immune defenses, and respond transiently to antimicrobial therapy. Thus, there is an unmet need for developing wound care products with both the biofilm-disrupting and antimicrobial activity for prevention and treatment of both acute and chronic wounds that involve biofilms. Furthermore, there is also a need for a non-antibiotic, non-toxic wound care or disinfectant composition that can be classified as generally recognized as safe (GRAS).

Where the wound is one caused intentionally, for example during a surgical procedure, there is a benefit from reducing biofilm-related microbes in preventing subsequent infection. For example, the compositions described herein can be used on surgical sites in advance of surgery as a wash for effectively pre-treating and doping the wound area.

The role of biofilms is discussed in U.S. Patent Pub. No. 2014/0275267, which notes that:
bacterial organisms which actively populate these common surfaces may form organized communities called biofilms. Bacterial cells forming these biofilm communities assume a biological phenotype that is markedly different than their corresponding planktonic (non-surface attached, or free-swimming) bacterial analogs . . . . Biofilms are a special form of contamination that have been shown to require as much 1000 times the dose of routine biocides in order to eradicate the microorganism contained within, as compared to planktonic forms.

One aspect of the problem is that biofilms have a wide range of pH. It had previously been viewed that pH was homogenous across microorganism environments at around pH 5 to 7. Recent studies, however, have shown that the pH range of biofilms is broader, ranging from about 3 to 8. In addition, biofilm pH is both variable and dynamic. In reacting to contact with certain treatment compositions, the pH of biofilm may change. The prior art has generally considered the problem of biofilms as a steady-state issue, assuming no variation, and not testing for such variation. Thus, the industry has been focused on applying compositions without addressing the true nature of the problem. This problem creates particular challenges with respect to compositions including weak acids, which ultimately rely on the process of protonation. Dynamic pH changes in biofilm can result in equilibrium in pH at the contact layer with weak acid solutions resulting in pH below the titration point.

Another aspect of the problem is that biofilms provide physical and chemical defenses for the microorganisms that must be breached in order to disrupt the living organism within. These defenses can include both the extracellular polymeric substances (EPS) layer of the biofilm and an inner layer of lipopolysaccharides (LPS). For example, studies have been cited suggesting that the intact LPS layer of enterobacteriaceae protected those organisms from anti-bacterial compositions.

Thus, microorganisms in biofilm colonies can be considered to have at least two distinct defense mechanisms: (1) the mechanism whereby the pH of the biofilm results in a change in pH at the composition contact layer that may be within the titration or inactivation point of the active ingredient, or to equilibrium; and (2) physical protections afforded by the EPS and LPS layers.

Current skin treatments and medicines are not generally suited for addressing a broad spectrum range of various types of microorganisms and are generally ineffective with respect to biofilms. Variations include physical and chemical composition of EPS/LPS, particularly in gram-negative bacteria, which can operate to make the penetration of biocides to be ineffective.

A composition seeking to be effective against biofilms and on a broad spectrum basis must adequately address these variations.

Examples of microorganisms associated with biofilms that are not effectively addressed by current skin treatments and medicines include the following:
Staphylococcus aureus is a gram-positive bacterium that is a common cause of infections. The organism is ubiquitous, with estimates of 30-40% of humans being colonized on mucosal surfaces. Illnesses caused by the organism range from benign infections, such as furuncles, to life-threatening illnesses, such as toxic shock syndrome (TSS)
Bacillus anthracis is a gram-positive rod that, through production of a cell surface capsule and other molecules and exotoxins, can cause serious illnesses. Such illnesses include skin, gastrointestinal, and pulmonary anthrax. This organism is characterized as a “category A select agent.”
Methicillin-resistant Staphylococcus aureus (MRSA) is a bacterium responsible for several difficult-to-treat infections in humans. It is also called oxacillin-resistant Staphylococcus aureus (ORSA). MRSA is any strain of S. aureus that has developed, through the process of natural selection, resistance to beta-lactam antibiotics, which include the penicillins (methicillin, dicloxacillin, nafcillin, oxacillin, etc.) and the cephalosporins.

A primary chemical interaction which can result in the breakdown of biofilms, LPS, and microorganisms, is protonation. Protonation is a fundamental chemical reaction and is a step in many stoichiometric and catalytic processes. Protonation and deprotonation occur in most acid-base reactions and are the core of most acid-base reaction theories.

For a given compound, protonation occurs at the point when the active molecule will donate the relevant proton, which is called the titration point. For example, the necessity of achieving the requisite composition pH and amine oxide protonation is discussed in U.S. Pat. No. 6,255,270, which discloses liquid cleaning compositions that include an amine oxide detergent, a quaternary disinfectant (quat), an acidifying agent, an effective amount of an electrolytic disinfecting booster, and an aqueous carrier.

The failure of certain cleaners and disinfectants to break down EPS and LPS defenses and eradicate microorganisms can result from insufficient or ineffective protonation. One problem is that protonation may require maintaining a sufficient difference in pH between the composition donating the protons and that of the surfactant layer in proximity to the microorganisms. In the event that the pH of the solution and the contact biomass is below the titration point for the active ingredient, protonation will reduce or cease and no longer effectively break down EPS and LPS defenses or disrupt the microorganisms therein.

Even where EPS and LPS defenses can be breached, it also is important to apply effective antimicrobial and biocidal substances to the microbes within. For example, as explained in U.S. patent Pub. No. 2013/0281532:
[m]ost bacterial pathogens initiate human illnesses from intact or damaged mucosal or skin surfaces. Many of these pathogens are acquired from other persons or animals, from endogenous sources, or from a myriad of environmental sources. Once in humans, pathogens colonize surfaces primarily as biofilms of organisms, defined as thin-films of organisms attached to host tissues, medical devices, and other bacteria through complex networks of polysaccharides, proteins, and nucleic acids. These bacteria may also exist as planktonic (broth) cultures in some host tissue environments, such as the bloodstream and mucosal secretions. Similarly, these potential pathogens may exist as either biofilms or planktonic cultures in a myriad of non-living environments.

US Pub. No. 2013/0281532 discusses compositions of glycerol monolaurate (GML), a naturally occurring glycerol-based compound that has previously been shown to have anti-microbial, anti-viral, and anti-inflammatory properties, to be applied as a topical composition in treating microbial infections and illnesses. GML is one chemical within the broader family of glycerol monoesters (GMEs). The class of GME compositions, including GML, have in certain circumstances been demonstrated to have potent antibacterial activity against gram-positive microorganisms and Bacillus anthracis. U.S. Pub. No. 2013/0281532 discloses that:
unlike most antibiotics which have single bacterial targets for antibacterial activities, GML appears to target many bacterial surface signal transduction systems nonspecifically through interaction with plasma membranes. GML also inhibits exotoxin production by gram-positive bacteria at GML concentrations that do not inhibit bacterial growth. These properties are shared with the antibiotic clindamycin, a protein synthesis inhibitor. GML is also virucidal for enveloped viruses, apparently through its ability to interfere with virus fusion with mammalian cells, and through GML's ability to prevent mucosal inflammation required for some viruses to penetrate mucosal surfaces. Studies demonstrate that GML is bactericidal for aerobic and anaerobic gram-positive bacteria in broth and biofilm cultures, GML exhibits greater bactericidal activity than lauric acid, and all forms of GML exhibit antibacterial activity. Additionally, GML is bactericidal for gram-negative bacteria with LOS instead of LPS, but GML becomes bactericidal for naturally GML-resistant Enterobacteriaceae by addition of agents that disrupt the LPS layer. Gram-negative anaerobes are susceptible to GML. Pseudomonas aeruginosa appear to be the most resistant bacteria tested, but these organisms are killed by GML at pH 5.0-6.0.

U.S. Pub. No. 2013/0281532 describes other studies demonstrating that GML and other compounds within the family of GME have potent bactericidal activity against many microorganisms causing human illnesses, including gram-positive bacteria (notably, gram-positive cocci); anaerobes; pathogenic clostridia; Candida; Gardnerella vaginalis; Staphylococcus aureus; and Streptococcus agalactiae. This includes both aerobes and anaerobes, and gram-positive, gram-negative, and non-gram-staining bacteria.

US patent application no. 0281532 concluded that:
it is thought that GML inhibits microbial infection through one or more of several mechanisms that include, but are not limited to, direct microbial toxicity; inhibiting entry of the infectious microorganism into the vertebrate cell; inhibiting growth of the microorganism; inhibiting production or activity of virulence factors such as toxins; stabilizing the vertebrate cells; or inhibiting induction of inflammatory or immunostimulatory mediators that otherwise enhance the infectious process.

The class of GME compositions, including GML, have been demonstrated to have potent antibacterial activity, as explained in recent NIH research reports, but subject to important perceived limitations. Schlievert, et al. Glycerol Monolaurate Antibacterial Activity in Broth and Biofilm Cultures, 10.1371/journal.pone.0040 350 (2012). GML's biocidal effect is substantially increased in low pH. However, NIH's recent research believed that “it is unlikely that GML will be used as an antibacterial agent as suspended in aqueous solutions do to its solubility limit of 100 μg/ml in aqueous solutions at 37° C.”

Thus there remains a need in the art for dermatologically safe and effective treatments for reducing or disrupting a microbial biofilm's EPS and LPS defenses in order to effectively deliver biocidal agents for treating, controlling or preventing skin conditions and diseases associated with microbial biofilms.

### SUMMARY

Aspects of the present invention features topical formulations that enhance the disruption of microbial biofilms and increase delivery of antimicrobial agents to the microbes within the microbial biofilms. In addition, provided herein are methods of applying the topical formulations for treating, controlling, or preventing skin conditions or diseases associated with microbial biofilms.

One aspect of the invention features a topical formulation system for the treatment, control, or prevention of a skin condition or skin disease associated with a microbial biofilm. In particular, the topical formulation system includes a set of reaction components. The first reaction component includes a cationic surfactant in an amount from about 10% wt to about 40% wt, a pharmaceutically acceptable carrier comprising one or more emulsifying agents, wherein a total amount of emulsifying agents is from about 5% wt to about 40% wt, and a dermatologically acceptable biocide in an amount of at least about 1% wt. The second reaction component includes an aqueous solution comprising one or more weak acids having a total amount of weak acids in a range from about 0.5% w/v to about 15% w/v, provided that the weak acids have a pH in a range from about 2 to about 6 and a first titration point pH. Additionally, the cationic surfactant of the first component has a pH of at least about 2 units greater than the first titration point pH of the weak acids. Furthermore, when the first and second reaction components are combined on a mucosal surface or skin surface comprising a microbial biofilm, a wetting layer is produced that increases protonation of water to produce hydronium increases delivery of the hydronium and the dermatologically acceptable biocide to the microbial biofilm thereby disrupting the microbial biofilm.

In certain embodiments, the second reaction component further comprises one or more emulsifying agents, wherein a total amount of emulsifying agents in the second reaction component is from about 0.2% w/v to about 10% w/v. In other embodiments, the one or more emulsifying agents in the second reaction component are skin permeable. In yet other embodiments, the one or more emulsifying agents in the second reaction component are selected from the group consisting of a glycerol monoester, sorbitan monolaurate, sodium stearoyl lactylate, polyoxyethylene (20) sorbitan monooleate, and any combination thereof.

In an embodiment, the cationic surfactant is a fatty acid salt or a saponified organic acid and wherein the pH of the one or more weak acids is less than about 3.5. In another embodiment, the cationic surfactant is potassium cocoate.

In some embodiments of the topical formulation, the one or more weak acids are selected from the group consisting of ascorbic acid, citric acid, salicylic acid, lactic acid, malic acid, tartaric acid, and any combination thereof. In others, the pharmaceutically acceptable carrier further comprises one or more nonaqueous oils or gels. In yet other embodiments, the one or more nonaqueous oils or gels are selected from the group consisting of olive oil, vegetable oil, petroleum jelly, and any combination thereof.

In one embodiment, the one or more emulsifying agents in the first reaction component are skin permeable. In another embodiment, the one or more emulsifying agents in the first reaction component are selected from the group consisting of sorbitan monolaurate, sodium stearoyl lactylate, polyoxyethylene (20) sorbitan monooleate, and any combination thereof. In yet another embodiment, the first reaction component is formulated for application as liquid, cream, or gel. In still other embodiments, the second reaction component is formulated for application as a spray or an aqueous gel. In some embodiments, the second reaction component is formulation for an aqueous gel comprising hyaluronic acid.

In one embodiment, the first reaction component further comprises one or more weak acids selected from the group consisting of salicylic acid, ascorbic acid, citric acid, and any combination thereof, wherein the total concentration of weak acids in the first reaction component is a least about 0.5% wt. In another embodiment, combining the reaction components on a mucosal surface or skin surface comprising a microbial biofilm produces a stable emulsified mixture in accordance with the hydrophilic-lipophilic balance system.

In one embodiment, the dermatologically acceptable biocide is a glycol monoester of the formula: R₁OCH2(OR₂)CH2OR₃ wherein R₁, R₂ and R₃ are individually H or a C6 to C22 acyl group. In another embodiment, the glycol monoester is selected from the group consisting of glycerol monocaprylate, glycerol monocaprate, glycerol monolaurate, glycerol monomyristate, and any combination thereof. In a particular embodiment, the glycol monoester is glycerol monolaurate at a concentration of greater than about 2% wt, and the cationic surfactant in the first reaction component is in an amount from about 15% wt to about 35% wt.

Another aspect of the invention features a method for treating, controlling or preventing a skin disease or skin condition associated with a microbial biofilm in an individual. The method includes the steps of providing an individual having a mucosal surface or skin surface comprising the microbial biofilm, applying a conditioning solution to the mucosal surface or skin surface of the individual, and applying an activating solution to the mucosal surface or skin surface of the individual. In the method, the conditioning solution includes (1) a cationic surfactant in an amount from about 15% wt to about 35% wt; (2) a pharmaceutically acceptable carrier comprising one or more skin permeable emulsifying agents, wherein the total amount of the emulsifying agents is from about 5% wt to about 40% wt; and (3) a dermatologically acceptable biocide in an amount of at least about 2% wt, wherein the biocide is a glycol monoester of the formula: R₁OCH2(OR₂)CH2OR₃ and where R₁, R₂ and R₃ are individually H or a C6 to C22 acyl group. The activating solution includes an aqueous solution comprising one or more weak acids having a total amount of weak acids ranging from about 0.5% w/v to about 15% w/v, provided that the one or more weak acids have a pH in a range from about 2 to about 6 and the cationic surfactant of the first component has a pH of at least about 2 units greater than the first titration point pH of the one or more weak acids. Furthermore, combining the conditioning solution and the activating solution at the mucosal surface or skin surface of the individual produces a wetting layer that increases protonation of water to produce hydronium and increases delivery of the hydronium and the dermatologically acceptable biocide to the microbial biofilm thereby disrupting the microbial biofilm and treating, controlling or preventing the skin disease or skin condition associated with the microbial biofilm in the individual. In other aspects, the conditioning solution and activating solution includes components as summarized above with respect to the first reaction component and second reaction component, respectively, of the topical formulation system.

In one embodiment of the method, the conditioning solution is into a cosmetic product, and the activating solution is incorporated into a cosmetic product remover. In another embodiment, the combination of the conditioning solution and the activating solution at the mucosal surface or skin surface of the individual produces a stable emulsified mixture in accordance with the hydrophilic-lipophilic balance system.

In some embodiments, the skin condition or skin disease is selected from the group consisting of atopic dermatitis, eczema, acne vulgaris, warts, wound infection, fungal skin disease, and viral skin disease. In another embodiment, the individual is a human or animal. In yet another embodiment, the activating solution is applied about 10 seconds to about 30 seconds after application of the conditioning solution.

Another aspect of the invention features a topical formulation for treatment, control or prevention of a skin condition or skin disease associated with a microbial biofilm that includes: (a) a cationic surfactant in an amount from about 1% w/v to about 5% w/v; (b) one or more skin permeable emulsifying agents, wherein a total amount of skin permeable emulsifying agents is from about 0.5% w/v to about 5% w/v; (c) a dermatologically acceptable biocide in an amount of at least about 0.1% w/v, wherein the dermatologically acceptable biocide is a glycol monoester of the formula: R₁OCH2(OR₂)CH2OR₃ wherein R₁, R₂ and R₃ are individually H or a C6 to C22 acyl group; and (d) at least one weak acid in an amount from about 0.5% w/v to about 15% w/v, provided that the at least one weak acid has a pH in a range from about 2 to about 6 and the cationic surfactant has a pH of at least about 2 greater than the first titration point pH of the at least one weak acid. In such aspects, a wetting layer is formed upon application of the topical formulation on a mucosal surface or skin surface comprising a microbial biofilm, wherein the wetting layer increases protonation of water to produce hydronium, and wherein the wetting layer increases delivery of the hydronium and the dermatologically acceptable biocide to the microbial biofilm thereby disrupting the microbial biofilm. In other aspects, the topical formulation is used in a method for treating, controlling or preventing a skin disease or skin condition associated with a microbial biofilm in an individual.

Another aspect of the invention features a multi-layered composition for enhanced delivery of hydronium and includes: (a) a surface layer on which is disposed a microbial biofilm; (b) a wetting layer that is disposed on the surface layer and that includes a cationic surfactant, one or more emulsifying agents, and a biocide having the formula R₁OCH2(OR₂)CH2OR₃ wherein R₁, R₂ and R₃ are individually H or a C6 to C22 acyl group; and (c) an emulsion layer that is disposed on the wetting layer and that includes water and one or more weak acids. In this aspect, the wetting layer and emulsion layer have a total weight, and: (i) the cationic surfactant is in an amount from about 10% wt to about 40% wt of the total weight; (ii) the one or more emulsifying agents are in an amount from about 5% wt to about 40% wt of the total weight; (iii) the biocide is in an amount of at least about 1% wt of the total weight; (iv) the one or more weak acids are an amount from about 0.5% wt to about 15% wt of the total weight; (vi) the one or more weak acids comprise a first titration point and have a pH in a range from about 2 to about 6; and (vii) the cationic surfactant has a pH of at least about 2 units greater than the first titration point pH of the one or more weak acids. Furthermore, the wetting layer increases protonation of water to produce hydronium, and wherein the wetting layer increases delivery of the hydronium and the biocide to the microbial biofilm thereby disrupting the microbial biofilm.

In some embodiments, the cationic surfactant is a fatty acid salt or a saponified organic acid and wherein the pH of the at least one weak acid is less than about 3.5. In other embodiments, the cationic surfactant is potassium cocoate, and wherein the one or more weak acids are selected from the group consisting of ascorbic acid, salicylic acid, citric acid, lactic acid, malic acid, tartaric acid, and any combination thereof. In yet other embodiments, the one or more emulsifying agents are selected from the group consisting of sorbitan monolaurate, sodium stearoyl lactylate, polyoxyethylene (20) sorbitan monooleate, and any combination thereof. In a particular embodiment, the surface is a skin surface or mucosal surface. In still other embodiments, the glycol monoester is selected from the group consisting of glycerol monocaprylate, glycerol monocaprate, glycerol monolaurate, glycerol monomyristate, and any combination thereof.

Other features and advantages of the invention will be understood by the detailed description, drawings and examples that follow.

### BRIEF DESCRIPTION OF THE DRAWINGS

The accompanying figures, which are incorporated in and constitute a part of this specification, illustrate several aspects and together with the description serve to explain the principles of the invention.

FIG. 1 is an illustration depicting the hyperprotonation layer at a microbial biofilm created by application of the compositions and systems of the invention. Three layers are depicted (from top to bottom of the illustration): (1) the emulsion, (2) the surfactant wetting layer, and (3) the microbial biomass. Lines between the three layers indicate (from top to bottom): the boundary layer created between the emulsion and the wetting layer, and the microbial biofilm. In the embodiment shown, the wetting layer is greater than pH 4.11, therefore above the lowest titration point of the citric acid disposed in the emulsion, causing titration and hyperprotonation through the wetting layer. Further, the titration event in the wetting layer does not consume the surfactant and therefore does not reach equilibrium, as would occur if there was direct contact with the biomass.

FIG. 2 is a graph depicting the hyperprotonation—pH balance and kill zone of an exemplary topical formulation. The y-axis indicates the weight percentage of citric acid, and the x-axis indicates the pH of the solution. In preferred embodiments, (1) the biocide (GME) concentration is greater than 500 micrograms per ml, (2) the surfactant concentration is greater than 0.5% w/v, (3) the steady state pH of the solution is not greater than the titration point of the acid, and (4) the pH of the surfactant mix (with emulsifier and GME) is at least 2 pH units higher than the lowest titration point of the acid.

FIG. 3 is a table depicting the effect of citric acid concentration on the change in pH of the surfactant and emulsifier composition for an embodiment of the invention. The composition of the exemplary topical formulation for the range of component values is balanced by distilled water (% w/v). The composition of GML in 0.50% emulsifiers is 750 μg/ml. The composition of GML in 0.75% emulsifiers is 1,125 μg/ml. The composition of GML in 1.00% emulsifiers is 1,500 μg/ml.

FIG. 4 is graph showing the log reduction of E. coli over time after contacting with an embodiment of a topical formulation. The y-axis indicates the log reduction of E. coli, and the x-axis indicates the amount of time elapsed in minutes.

FIG. 5 is graph showing the log reduction of Salmonella spp. over time after contacting with an embodiment of a topical formulation. The y-axis indicates the log reduction of Salmonella spp., and the x-axis indicates the amount of time elapsed in minutes.

FIG. 6 is graph showing the log reduction of S. aureus over time after contacting with an embodiment of a topical formulation. The y-axis indicates the log reduction of S. aureus, and the x-axis indicates the amount of time elapsed in minutes.

FIG. 7 is graph comparing the log reduction of Salmonella spp. over time after contacting with an embodiment of a topical formulation (circle) as compared to benzalkonium chloride (triangle), bleach (diamond), and lye (square). The y-axis indicates the log reduction of Salmonella spp., and the x-axis indicates the amount of time elapsed in minutes.

FIG. 8 are pictures of an individual with cystic acne after 1, 2, 3, and 4 days of treatment with an exemplary embodiment of the topical formulation system.

### DETAILED DESCRIPTION

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Composition, formulation, and/or reaction components may have several known functions, but may be selected and identified for a particular function (e.g., a buffer). However, as one skilled in the art may appreciate, the component may be performing multiple functions within the composition, formulation, and or reaction (e.g., a surfactant may function as a wetting agent and as an emulsifier).

All percentages expressed herein are by weight of the total volume of the composition or mixture unless expressed otherwise. All ratios expressed herein are on a weight per volume (% w/v) or weight per total weight (% wt or wt %) basis as indicated.

Ranges may be used herein in shorthand, to avoid having to list and describe each value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a method” or “a microbe” includes a plurality of such “methods”, or “microbes.” Likewise the terms “include”, “including”, and “or” should all be construed to be inclusive, unless such a construction is clearly prohibited from the context. Similarly, the term “examples,” particularly when followed by a listing of terms, is merely exemplary and illustrative and should not be deemed exclusive or comprehensive.

The term “comprising” is intended to include embodiments encompassed by the terms “consisting essentially of” and “consisting of”. Similarly, the term “consisting essentially of” is intended to include embodiments encompassed by the term “consisting of.”

The methods and compositions and other advances disclosed herein are not limited to particular equipment or processes described herein because such equipment or processes may vary. Further, the terminology used herein is for describing particular embodiments only and is not intended to limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

The term “about” refers to the variation in the numerical value of a measurement, e.g., temperature, parts per million (ppm), pH, concentration, volume, etc., due to typical error rates of the device used to obtain that measure. In one embodiment, the term “about” means within 5% of the reported numerical value.

The term “antimicrobial” refers effectiveness in preventing, inhibiting, or arresting the growth or pathogenic effects of a microorganism.

The term “biocide” refers to a chemical substance or microorganism which can deter, render harmless, or exert a controlling effect on an organism by chemical or biological means. “Biocides” are commonly used in medicine, agriculture, forestry, and industry. Biocidal substances and products are also employed as anti-fouling agents or disinfectants under other circumstances: chlorine, for example, is used as a short-life biocide in industrial water treatment but as a disinfectant in swimming pools. Many biocides are synthetic, but a class of natural biocides are derived from, e.g., bacteria and plants. As used herein, “biocide” can refer to a pesticide (e.g., fungicides, herbicides, insecticides, algicides, molluscicides, miticides and rodenticides) or an antimicrobial agent (e.g., germicides, antibiotics, antibacterials, antivirals, antifungals, antiprotozoals and antiparasites).

The terms “biofilm” and “microbial biofilm” refer to any group of microorganisms in which cells stick to each other on a surface. These adherent cells are frequently embedded within a self-produced matrix of extracellular polymeric substance (EPS). As used herein, “microbial biofilm” may also refer to and/or include a group of viral particles.

The terms “extracellular polymeric substances” and “EPS” refer to a generally sticky rigid structure of polysaccharides, DNA, and other organic contaminants that are produced and embedded on the surface of a microbial biofilm. A biofilm layer is anchored firmly to a surface and provides a protective environment in which microorganisms grow. Bacteria, viruses, yeasts, molds, and fungi contained in the biofilms can become dormant and therefore reduce their uptake of nutrients and/or antimicrobial agents.

The term “decontamination” refers to the neutralization or removal of dangerous substances from an area, object, surface, person, or animal.

The term “dermatologically-acceptable,” as used herein, refers to compounds or compositions suitable for use in contact with tissues (e.g., the skin) without undue toxicity, incompatibility, instability, irritation, allergic response, and the like. Likewise, the term “pharmaceutically acceptable” as used herein to refer to, e.g., a carrier, means a material, diluent, or vehicle that can be applied to skin or mucosal surfaces without undue toxicity, irritation, or allergic reaction.

The term “disinfectant” refers to antimicrobial agents that are applied to non-living objects to destroy microorganisms that are living on the objects and works by destroying the cell wall of microbes or interfering with microbial metabolism. Disinfection does not necessarily kill all microorganisms, especially resistant bacterial spores, and it is typically less effective than sterilization, which is an extreme physical and/or chemical process that kills all types of life. “Disinfectants” are different from other antimicrobial agents, such as antibiotics which destroy microorganisms within the body, and antiseptics which destroy microorganisms on living tissue. “Disinfectants” are also different from biocides—the latter are intended to destroy all forms of life, not just microorganisms.

As used herein, the term “eczema” refers to a disorder of the skin characterized by scaled or crusty patches of skin, often accompanied by redness, blistering, and itching, and perhaps blemishes or skin lesions. The term “eczema” includes a variety of conditions, including, but not limited to, “atopic eczema,” “contact eczema,” “seborrheic eczema,” “nummular eczema,” “neurodermatitis,” “stasis dermatitis,” and “dyshidrotic eczema.” “Atopic eczema” refers to a hereditary predisposition for inflammation in the skin. “Contact eczema” is a general term for an inflamed skin condition caused by contact of the skin to an irritant or allergen. Hence, specific forms of contact eczema include allergic contact eczema and irritant contact eczema. “Seborrheic eczema,” also known as seborrhea or seborrheic dermatitis, refers to eczema predominantly of the scalp, but may affect other parts of the body. “Seborrheic eczema” is often associated with dandruff, scaling, and redness. “Nummular eczema,” also known as nummular eczematous dermatitis or discoid eczema, is characterized by coin-shaped lesions on the skin. The cause of the lesions may be dry skin in low humidity environments, or bacterial infections that induce hypersensitivity in the skin. “Neurodermatitis” refers to a chronic type of eczema, characterized by raised, rough, itchy patches of skin, typically on the neck, wrist, and ankles. Possible causes of “neurodermatitis” include sensitization of the skin over time by an external agent, or by stress, anxiety, dry skin, or infection. “Stasis dermatitis” refers to a condition characterized by a red, itchy rash on the lower legs, which may form a serious condition causing swelling of the legs. The common cause of “stasis dermatitis” is poor blood flow from the legs to the heart. “Dyshidrotic eczema,” also known as dyshidrosis, or pompholyx, refers to a condition characterized by the formation of small blisters on the skin (typically on the hands and feet) that cause intense itching and may form into an intensely itchy rash. A possible cause of “dyshidrotic eczema” is an inherited allergic response in the skin.

The term “sanitizer” refers to substances that simultaneously clean and disinfect.

The term “eradication” means the complete destruction of a microbe colony, as demonstrated in testing of microbes in real world settings such as biofilms, such that no further microbes are detected in testing following a period of application of at least 18 minutes.

The term “hydronium” is the common name for the aqueous cation H₃O⁺, the type of oxonium ion produced by protonation of water. It is the positive ion present when an Arrhenius acid is dissolved in water, as Arrhenius acid molecules in solution give up a proton (a positive hydrogen ion, H⁺) to the surrounding water molecules (H₂O). It is the presence of hydronium ion relative to hydroxide that determines a solution's pH. The molecules in pure water auto-dissociate into “hydronium” and hydroxide ions in the following equilibrium: 2 H₂O OH⁻+H₃O⁺ In pure water, there is an equal number of hydroxide and hydronium ions, so it has a neutral pH of 7. A pH value less than 7 indicates an acidic solution, and a pH value more than 7 indicates a basic solution.

The terms “hydrophilic-lipophilic balance” and “HLB” when referring to a surfactant is a measure of the degree to which it is hydrophilic or lipophilic, determined by calculating values for the different regions of the molecule.

The terms “lipopolysaccharides” and “LPS” are also known as lipoglycans and endotoxin, and refer to large molecules consisting of a lipid and a polysaccharide composed of O-antigen, an outer core and an inner core joined by a covalent bond. “LPS” are found in the outer membrane of Gram-negative bacteria and elicit strong immune responses in animals.

The terms “microbe” and “microorganism” are used herein to mean any bacteria, virus, or fungus, including, but not limited to, Staphylococcus aureus, Streptococcus (e.g., S. pyogenes, S. agalacticae or S. pneumoniae), Haemophilus influenzae, Pseudomonas aeruginosa, Gardnerella vaginalis, Enterobacteriacae (e.g., Escherichia coli), Clostridium perfringens, Chlamydia trachomatis, Candida albicans, Human Immunodeficiency Virus (HIV), or Herpes Simplex Virus (HSV).

The terms “methicillin-resistant Staphylococcus aureus” and “MRSA” refer to a bacterium responsible for several difficult-to-treat infections in humans. It is also called oxacillin-resistant Staphylococcus aureus (ORSA). “MRSA” is any strain of Staphylococcus aureus that has developed, through the process of natural selection, resistance to beta-lactam antibiotics, which include the penicillins (e.g., methicillin, dicloxacillin, nafcillin, oxacillin, etc.) and the cephalosporins. Strains unable to resist these antibiotics are classified as methicillin-sensitive Staphylococcus aureus, or MSSA. The evolution of such resistance does not cause the organism to be more intrinsically virulent than strains of S. aureus that have no antibiotic resistance, but resistance does make MRSA infection more difficult to treat with standard types of antibiotics and thus more dangerous.

The term “protonation” refers to the transfer of a proton to a molecule, group, or atom, such that a coordinate bond to the proton is formed. “Protonation” is a fundamental chemical reaction and a step in many stoichiometric and catalytic processes. Some ions and molecules can undergo more than one “protonation” and are labeled polybasic or polyprotic, which is true of many biological macromolecules. “Protonation” and deprotonation occur in most acid-base reactions; they are the core of most acid-base reaction theories.

As used herein, the term “skin condition” is intended to be used in its broadest sense, including but not limited to, all of the specific conditions referenced herein with respect to all types of human and animal skin tissues. While they are discussed with reference to human skin, the term is not intended to be so limited, but also encompasses similar or analogous conditions affecting livestock, pets, or other animals, including those which may be addressed in veterinary and animal clinical settings.

The term “sterilization” refers to any process that removes, eliminates, or kills all forms of life, including transmissible agents (such as fungi, bacteria, viruses, spore forms, etc.) present in a specified region, such as a surface, a volume of fluid, medication, or in a compound such as biological culture media. “Sterilization” can be achieved with one or more of the following: heat, chemicals, irradiation, high pressure, and filtration. “Sterilization” is distinct from disinfection, sanitization, and pasteurization in that “sterilization” kills or inactivates all forms of life.

The term “surfactant” refers to a compound that lowers the surface tension (or interfacial tension) between two liquids or between a liquid and a solid. “Surfactants” may act as detergents, wetting agents, emulsifiers, foaming agents, and dispersants.

The term “titration curve” refers to a curve in the plane whose x-coordinate is the volume of titrant added since the beginning of the titration, and whose y-coordinate is the concentration of the analyte at the corresponding stage of the titration (in an acid-base titration, the y-coordinate is usually the pH of the solution).

The term “topical,” as used herein, refers to the application of the formulations to any skin or mucosal surface. “Skin surface” refers to the protective outer covering of the body of a vertebrate, generally comprising a layer of epidermal cells and a layer of dermal cells. A “mucosal surface,” as used herein, refers to a tissue lining of an organ or body cavity that secretes mucous, including, but not limited to, oral, vaginal, rectal, gastrointestinal, and nasal surfaces. In one embodiment, the formulations of the invention are administered topically to the teeth and gum, skin, nasal, or vaginal areas.

The term “topically applying” means directly laying on or spreading on any skin or mucosal tissue, e.g., by use of hands or an applicator such as a wipe, puff, roller, or spray.

The term “weak acid” refers to an acid with pH above about 2.0 and below about 7.0. All pH values herein are measured in aqueous systems at 25° C. (77° F.).

All patents, patent applications, publications, technical and/or scholarly articles, and other references cited or referred to herein are in their entirety incorporated herein by reference to the extent allowed by law, as if separately set forth herein. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Although the foregoing specification and examples fully disclose and enable the present invention, they are not intended to limit the scope of the invention, which is defined by the claims appended hereto. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

This invention springs in part from the inventor's identification of the interrelation of several specific problems associated with microbial biofilm and skin conditions. First, as physical structures around microbes, biofilms inhibit access and thereby defend against application of treatments. Second, when contacted by a treatment solution, biofilms operate to create a layer of pH equilibrium that inhibits biochemical reactions that would disrupt tenant microbes. Third, as result of the first two factors, biofilms are virtually always successful in preserving at least small pockets of microbes after contact with biocides. Because microorganisms reproduce very rapidly, any reduction in skin impact will be temporary and overtaken as the population growth resumes.

To effectively solve these challenges, exemplary compositions and formulations provided herein provide a concentration of highly-effective biocide, such as the natural and non-toxic GME antimicrobial biocides, as well as an efficient delivery mechanism that incorporates emulsifier ingredients to act as a carrier for delivery of the antimicrobial biocides to the microbial biofilms to enable the biocides to reach the microbial biofilm at higher concentration thereby increasing the disruption of the microbial biofilm. In addition, by combining a surfactant and a weak acid, the composition of the formulation operates to create a zone of hyperprotonation in what effectively is a membrane enveloping all or part of the biofilm structure. In other words, the present compositions create an enveloping membrane around the microbial biofilms that disrupts and neutralizes their defenses, and delivers safe, natural antibacterial and anti-viral active ingredients, such as the GME antimicrobial biocides. The enveloping membrane can be described as a “hydronium engine” that osmotically or, in some embodiments, through emulsion, delivers both hydronium and GME to the microbial biomass.

In one aspect, the invention features compositions and methods that are of greater efficacy in disrupting biofilms associated with common skin diseases and conditions. In such aspect, the invention disclosed herein incorporates a newly discovered understanding of the relationship of pH of the treatment composition and the dynamic pH of biofilms and microorganisms within biofilms. In particular embodiments, the composition is a topical formulation that includes a surfactant, one or more emulsifying agents, a dermatologically acceptable biocide, and a weak acid. As one skilled in the art will appreciate, surfactants are capable of functioning as emulsifiers. However, while not intending to disclaim any particular function, suitable components for use in the present formulations are chosen and identified for a particular function, e.g., surfactant, wetting agent, emulsifier, spreading agent, detergent, dispersant, or foaming agent, despite the fact that the particular component may serve some or all of these functions.

In some embodiments, one or more emulsifying agents serve as a pharmaceutically acceptable carrier that permits safe application to the skin surface or mucosal surface of an individual. In other embodiments, the pharmaceutically acceptable carrier is a mixture of components that include one or more emulsifying agents and/or a nonaqueous oil or gel that enables the topical application of the composition to the skin or mucosal surface of an individual, the delivery of which serves to disrupt the microbial biofilm and treat, control or prevent a skin disease or skin condition that is associated with or caused by that microbial biofilm. In such embodiments, the topical formulation includes a surfactant, pharmaceutically acceptable carrier (including one or more emulsifying agents), a dermatologically acceptable biocide, and a weak acid. Once applied to the skin surface or mucosal surface of an individual, the formulation produces a wetting layer at the surface of the microbial biofilm to increase the delivery and efficacy of biofilm disrupting agents, such as hydronium produced at the wetting layer and the biocide component, as will be explained in more detail below.

In other aspects, the composition is a topical formulation applied as a two-step or two-component system. In such aspects, the system includes a set of reaction components. The first component includes at least the surfactant, pharmaceutically acceptable carrier (e.g., emulsifying agents), and a dermatologically acceptable biocide; albeit at a greater concentration as compared to a single solution formulation. Further, the second component includes one or more weak acids in an aqueous solution, which may include one or more additional components, e.g., emulsifying agents. In some embodiments, the first component is applied to the skin surface or mucosal surface of an individual having, or at risk of having, a skin condition or skin disease associated with a microbial biofilm. The second component is then applied to the skin surface or mucosal layer of the individual and, when combined or mixed with the first component, forms a wetting layer at the surface of the biofilm that increases the delivery and efficacy of biofilm disrupting agents, such as hydronium produced at the wetting layer and the biocide component, as will be explained in more detail below. The first component and/or the second component may be formulated as, e.g., a liquid, cream, mist or gel, and applied by use of the hands or by using an applicator, such as a wipe, puff, roller or spray. In a preferred embodiment, the first component is formulated as a liquid, cream, or gel, and the second component is formulated as a spray or mist.

The components and agents of topical formulations suitable for use herein will now be explained in further detail.

### Single Solution Topical Formulations

As noted above, a surfactant is employed to achieve a wetting layer at the surface of the biofilm. This surface wetting creates the equivalent of a membrane, so that osmotic pressure continues the flow of aqueous solution through the wetting layer. In preferred embodiments, the topical formulation includes one or more cationic surfactants (e.g., saponified organic acids, synthetic detergents, or a combination thereof) having a pH equal to or greater than 7. In more preferred embodiments, the cationic surfactant has a pH of at least 9. In a most preferred embodiment, the cationic surfactant is any potassium or sodium salt soap derived from one or more organic acids. In one particular non-limiting embodiment, the cationic surfactant is potassium cocoate. A suitable concentration of the cationic surfactant in the topical formulation is between about 0.5% w/v to about 10% w/v; preferably, between about 1% w/v to about 5% w/v, e.g., about 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5% w/v. In other embodiments, the concentration of the cationic surfactant in the topical formulation is between about 5 g/L to about 100 g/L; preferably, between about 10 g/L to about 50 g/L, e.g., 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, or 50 g/L.

In addition to a surfactant, the topical formulations described herein may include one or more weak acids. As one skilled in the art will appreciate, weak acids typically function in solution as buffering agents and can affect the pH of the wetting layer (e.g., maintaining a low pH of the wetting layer). Weak acid buffering agents suitable for use herein typically include organic acids having a pH between about 2 and 7. Preferably, the weak acid will have a pH less than or equal to 3.5; more preferably less than or equal to 3.0. Non-limiting exemplary weak acids include, but are not limited to, citric acid (pH of about 2.2), lactic acid (pH of about 2.4), malic acid (pH of about 2.2), tartaric acid (pH of about 2.2), salicylic acid (pH of about 2.4), ascorbic acid (pH of about 3.4), and any combination of such weak acids. A suitable concentration of the weak acid, or combination of weak acids, in the topical formulation is between about 0.2% w/v to about 20% w/v; preferably, between about 0.5% w/v to about 15% w/v, e.g., about 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, or 15.0% w/v. In other embodiments, the concentration of the weak acid(s) in the topical formulation is between about 2 g/L to about 200 g/L; preferably, between about 5 g/L to about 150 g/L, e.g., 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, or 150 g/L.

Once the topical formulation is contacted to a surface, such as a skin surface or mucosal surface, the surfactant will form a wetting layer. If a cationic surfactant is used, the pH of the wetting layer will be much higher than that of the weak acid. As the weak acid and surfactant mix, the pH of the wetting layer changes depending on the pH difference between the weak acid and the surfactant. As one skilled in the art would readily appreciate, in an acid-base titration, the titration curve reflects the strength of the corresponding acid and base. For a strong acid and a strong base, the curve will be relatively smooth and very steep near the equivalence point. Because of this, a small change in titrant volume near the equivalence point results in a large pH change and many indicators would be appropriate (for instance litmus, phenolphthalein or bromothymol blue). If one reagent is a weak acid or base and the other is a strong acid or base, the titration curve is irregular and the pH shifts less with small additions of titrant near the equivalence point. More complex titration curves are produced by mixing polyprotic weak acids with a strong base. For instance, if a cationic surfactant is used with a high pH, such as potassium cocoate (pH of about 10) in addition to a polyprotic weak acid, such as oxalic acid or citric acid, the weak-acid/surfactant mixture may produce an irregular titration curve, the titration curve will be irregular having more than one inflection, or titration, points. The titration point, or first titration point for polyprotic acids, can therefore be used in some embodiments to select a suitable weak acid.

It is preferable that the weak acids used in the topical formulations of the present invention have a first titration point that is lower than the pH of the surfactant. In some embodiments, suitable weak acids will have a first titration point pH of less than about 6.0. In other embodiments, the weak acid in the topical formulation will have a first titration point pH of less than about 5.0; preferably less than about 4.0. In particular embodiments, the surfactant used in the topical formulation is a cationic surfactant having a pH that is higher than the first titration point of the weak acid present in the topical formulation. In more preferred embodiments, the cationic surfactant will have a pH that is at least 2.0 higher than the first titration point of the weak acid; most preferably, at least 3.0 higher.

As one skilled in the art will appreciate, while their principal function in the formulations described herein is to lower the pH, some weak acids may serve additional therapeutic functions. In particular, salicylic acid and ascorbic acid are well known to have therapeutic benefits in the treatment of skin. For instance, salicylic acid at a concentration of at least about 0.5% w/v has been approved by the FDA for the treatment of acne, eczema, and warts. As such, some embodiments include from about 0.5% w/v to about 10% w/v salicylic acid or from about 0.5% w/v to about 10% w/v ascorbic acid. In other embodiments, the topical formulations provided herein include at least about 1% w/v salicylic acid or ascorbic acid. In yet other embodiments, the topical formulation will include two or more of citric acid, salicylic acid, and ascorbic acid, each having a concentration of at least about 0.5% w/v; preferably, each having a concentration of at least about 1% w/v.

In some embodiments, the topical formulation includes a biocide. Biocides particularly suitable for use in the topical formulations disclosed herein include antimicrobial biocides, such as germicides, antibiotics, antibacterials, antivirals, antifungals, antiprotozoals, and antiparasites. In certain embodiments, the biocide is a glycerol monoester (GME). GMEs are particularly suitable for use as biocides since they can also function as emulsifiers, analgesics, and anti inflammatory agents in the topical formulations thereby providing a therapeutic benefit in addition to acting as a microbial biocide. See, e.g., U.S. 2013/0281532; Schlievert, et al. Glycerol Monolaurate Antibacterial Activity in Broth and Biofilm Cultures, 10.1371/journal.pone.0040 350 (2012), the entire contents of each of which are incorporated by reference herein.

In preferred embodiments, the GME is glycerol linked to a C6-C22 acyl group (e.g., C(═O)C5-C21 alkyl, wherein the alkyl is branched or unbranched, saturated or unsaturated). In these embodiments, the GME suitable for use has the formula R₁OCH₂(OR₂)CH₂OR₃, wherein R₁, R₂, and R₃ can either be a hydrogen (H) or a C6 to C22 acyl group. In some embodiments, the acyl group is branched or unbranched, saturated or unsaturated. In other embodiments, the acyl group is unbranched and saturated. In preferred embodiments, the acyl group is derived from a fatty acid, e.g., caprylic acid, capric acid, lauric acid, myristic acid, palmitic acid, stearic acid, arachidic acid, or behenic acid. In particular embodiments, the GME is glycerol monocaprylate (C8), glycerol monocaprate (CIO), glycerol monolaurate (CI 2, “GML”), or glycerol monomyristate (CI 4). GMEs, including GML, have been determined by the U.S. Environmental Protection Agency to be non-toxic (see 69 FR 34937) and have been listed in the Generally Recognized as Safe (GRAS) substances by the U.S. Food and Drug Administration. Indeed, GML occurs naturally in honey and human breast milk. GML and related compounds have been previously disclosed in U.S. patent application Ser. No. 10/579,108 (filed Nov. 10, 2004) and Ser. No. 11/195,239 (filed Aug. 2, 2005), the disclosures of each of which are herein incorporated by reference in their entireties. In some embodiments, the concentration of the biocide in the topical formulation is from about 10 μg/ml to about 10,000 μg/ml. In preferred embodiments, the concentration of the biocide is at least about 0.05% w/v; more preferably, at least about 0.1% w/v; most preferably, it is at least about 0.15% w/v. In some embodiments, the concentration of the biocide in the topical formulation is at least about 10 μg/ml; preferably, it is at least about 100 μg/ml; more preferably it is at least about 500 μg/ml; most preferably, it is at least about 1,000 μg/ml. In a non-limiting exemplary embodiment, a topical formulation is provided that includes about 1,500 μg/ml biocide, e.g., GML.

In an embodiment, the topical formulation includes a pharmaceutically acceptable carrier. In some embodiments, the pharmaceutically acceptable carrier is one or more emulsifying agents. In other embodiments, the pharmaceutically acceptable carrier includes one or more emulsifying agents and one or more additional agents, including, but not limited to, one or more nonaqueous oils or gels. For instance, in some embodiments, the pharmaceutically acceptable carrier includes olive oil, vegetable oil, and/or petroleum jelly. In preferred embodiments, the emulsifying agents are skin permeable. In such embodiments, the emulsifying agents suitable for use herein include, but are not limited to, sorbitan monolaurate (Polysorbate 20), sodium stearoyl lactylate, polyoxyethylene (20) sorbitan monooleate (Polysorbate 80), or any combination thereof. In some embodiments, the total concentration of emulsifying agents in the topical formulation are from about 0.2% to about 10% w/v; preferably, from about 0.5% w/v to about 5% w/v, e.g., about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5% w/v. In other embodiments, the concentration of the emulsifying agents in the topical formulation is between about 2 g/L to about 100 g/L; preferably, between about 5 g/L to about 50 g/L, e.g., 5, g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, or 50 g/L.

Other components may be included in the compositions and formulations disclosed herein. In some embodiments, the topical formulation includes thickeners such as synthetic polymers, fatty acids, fatty acid salts and esters, fatty alcohols, modified celluloses or modified mineral materials. In such embodiments, the thickeners can also be employed with liquid carriers to form spreadable pastes, gels, ointments, soaps, and the like, for application directly to the skin of the user. Examples of useful dermatological compositions which can be used to deliver the actives in the topical formulations to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), the content of each of which is incorporated herein by reference in their entireties.

Topical formulations of the present invention include any combination of the components described above and in any of the above-described concentrations. When the topical formulation is applied to the skin surface or mucosal surface of an individual having, or at risk for having, a skin condition or skin disease associated with a microbial film, the surfactant forms a membrane-like wetting layer at the surface of the microbial biofilm and maintains the osmotic pressure flow of aqueous solution through the wetting layer. In addition, topical formulations containing surfactants with a pH that is higher than that of the weak acid and, in particular, cationic surfactants having a pH of greater than 7, produce a wetting layer with an elevated pH, such that the pH of the wetting layer exceeds the first titration point of the weak acid component. By combining a weak acid with the wetting layer in proper pH-titration point balance, the invention maintains continuous and enhanced protonation in the surfactant layer, which results in ongoing creation of hydronium at the surface of the EPS as protons are donated from the weak acid to water. It is a catalytic process. Additionally, the surfactant compounds at the wetting layer and maintaining the membrane pH levels are not consumed in the process.

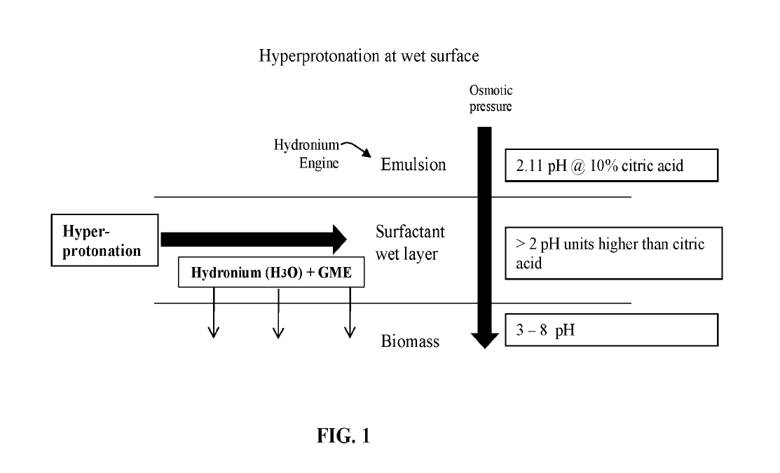

Shown in FIG. 1 is an illustration of a preferred embodiment of the wetting layer formed when the topical formulation is applied to a surface. In this non-limiting embodiment, three layers are depicted: (1) the emulsion, (2) the surfactant wetting layer, and (3) the microbial biomass. As shown in FIG. 1, the wetting layer has a pH greater than 4.11 and therefore above the lowest titration point of the citric acid disposed in the emulsion, causing titration and hyperprotonation through the wetting layer. Further, the titration event in the wetting layer does not consume the surfactant and therefore does not reach equilibrium, as would occur if there was direct contact with the biomass. The three-layer structure produced by the topical formulations described herein can be described as a “hydronium engine” as the hyperprotonation of water from acid in the wetting layer increases the hydronium available for delivery to the microbial biomass. Further, the hydronium delivery and the osmotic gradient across the layer gives the wetting layer characteristics similar to semipermeable membranes.

The topical formulations described herein have increased efficacy due, in part, to the ability of the topical formulation to disrupt the defenses of microbial biofilms that are formed by microbes in response to many factors, including cellular recognition of specific or non-specific attachment sites on a surface, nutritional cues, or in some cases, by exposure of planktonic cells to sub-inhibitory concentrations of antibiotics. When a cell switches to the biofilm mode of growth, it undergoes a phenotypic shift in behavior in which large suites of genes are differentially regulated.

Important to the microbial biofilm's defenses are the presence of EPS and LPS molecules. LPS is the major component of the outer membrane of Gram-negative bacteria, contributing greatly to the structural integrity of the bacteria, and protecting the membrane from certain kinds of chemical attack. LPS also increases the negative charge of the cell membrane and helps stabilize the overall membrane structure. It is of crucial importance to gram-negative bacteria, whose death results if it is mutated or removed. LPS induces a strong response from normal animal immune systems and has also been implicated in non-pathogenic aspects of bacterial ecology, including surface adhesion, bacteriophage sensitivity, and interactions with predators such as amoebae.

EPS are high-molecular weight compounds secreted by microorganisms into their environment. EPS establish the functional and structural integrity of biofilms, and are considered the fundamental component that determines the physiochemical properties of a biofilm. EPS are mostly composed of polysaccharides (exopolysaccharides) and proteins, but include other macro-molecules such as DNA, lipids, and humic substances.

One of the benefits of the present topical formulations is that they enhance protonation at the microbial biofilm surface, which disrupts the LPS and EPS defenses. Protonation is the addition of a proton to an atom, molecule, or ion. The proton is the nucleus of the hydrogen atom, and the positive hydrogen ion, H+, consists of a single proton. An example of protonation is the formation of the ammonium group NH₄+ from ammonia, NH₃. Protonation often occurs in the reaction of an acid with a base to form a salt. Protonation differs from hydrogenation in that during protonation a change in charge of the protonated species occurs, whereas the charge is unaffected during hydrogenation. Protonations are often rapid, in part because of the high mobility of protons in water. The rate of protonation is related to the acidity of the protonating species, in that protonation by weak acids is slower than protonation of the same base by strong acids. The rates of protonation and deprotonation can be especially slow when protonation induces significant structural changes.

The composition of the topical formulation effectively augments or hyper-charges the ongoing impact of the protonation by the weak acid—what is defined by this application as “hyperprotonation.” In hyperprotonation, the pH in the wetting layer remains above the titration point of the acid and thus maintains ongoing production of hydronium (heavy water H₃O) in a protonation process. By providing compositions that maintain the pH at the biofilm layer above the first titration point of the weak acid within the composition, the invention enables protonation to continue to occur, such that the microbial biofilm's EPS and LPS defenses are effectively breached. Importantly, the lower pH on the target surface is not an impediment to ongoing protonation which occurs in the wetting layer.

Another key aspect of microbial biofilm defenses is their ability to establish a pH equilibrium at the surface layer that effectively block lower pH solutions from reaching the biomass. Disrupting these defenses through hyperprotonation reduces the pH in the microbial biofilm, thereby increasing the potency of a microbial biocide to kill microbes by as much as eight orders of magnitude. See, e.g., Glycerol Monolaurate and Biofilm Technical Paper, U.S. National Institutes of Health (2012), the content of which is incorporated herein by reference in its entirety.

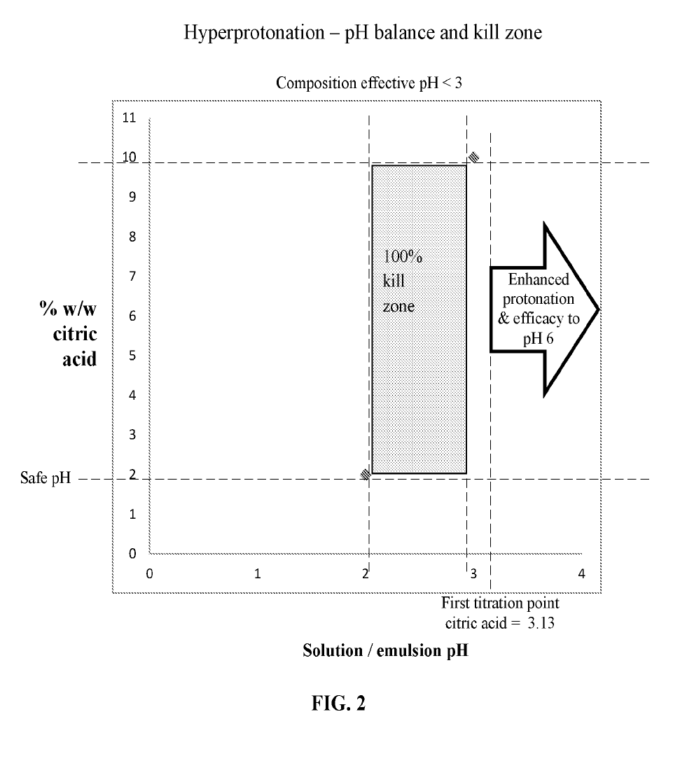

Shown in FIG. 2 is a depiction of the kill zone of an exemplary topical formulation. In FIG. 2, the biocide (e.g., GME) concentration is greater than 500 μg/ml, the surfactant concentration is greater than about 0.5% w/v, the steady state pH of the solution is not greater than the titration point of the acid, and the pH of the surfactant mix (with emulsifier and GME) is at least 2 pH units higher than the titration point of the acid.

The topical formulations provided herein can be applied to the skin or mucosal surface of an individual having a skin condition or disease, or is at risk for developing a skin condition or disease, associated with a microbial biofilm. In one embodiment, the topical formulation is applied directly to the skin surface or mucosal surface as a liquid formulation. In other embodiments, the topical formulation is applied as a cream or gel. In yet other embodiments, it is sprayed onto the skin surface or mucosal surface of the individual.

### Multi-Component Topical Formulation Systems

In another embodiment, the compositions presented herein are administered as a multi-component or multi-formulation system. Multi-component systems allow for the application of even higher concentrations of biocides for more effective antimicrobial properties. In one particular embodiment, provided herein is a topical formulation system that comprises two separate reaction components, or solutions, that are applied simultaneously or sequentially to the skin surface or mucosal surface. In a preferred embodiment, the two reaction components are applied sequentially.

In one embodiment, the topical formulation system comprises a conditioning solution, which includes a surfactant and biocide mixture, and an activating solution, which contains the proton donor, or activator (i.e., weak acid). The conditioning solution is first applied directly to the skin or mucosal surface of the individual to condition the skin or mucosal surface and provide an input or doping of the biocide. The activating solution is then applied to provide the weak acid proton donor. The combination of the conditioner solution and the activator solution provide the hyperprotonation and enhanced delivery of hydronium and biocide to the microbial biofilm. The hyperprotonation works to disrupt the EPS and LPS defenses of the microbial biofilm while the biocide disrupts the microbes themselves. Thus, the biocidal efficacy of the topical formulation is increased. The two-component system enhances stability and enhances antimicrobial effectiveness by delivering larger quantities of biocide (e.g., GML) transdermally prior to activation. In some embodiments, the conditioning solution is formulated as a liquid, cream, or gel and applied directly to the skin, while the activating solution is formulated as an aqueous spray that is applied to the skin using, e.g., an art-standard spray bottle.

In an embodiment, the conditioning solution includes a surfactant, such as a cationic surfactant, a pharmaceutically acceptable carrier, and a biocide. In such embodiment, the activating solution includes an aqueous solution containing at least the weak acid. In yet other embodiments, the conditioning solution includes a surfactant, such as a cationic surfactant, one or more emulsifiers, a biocide (e.g., a GME that also functions as a skin permeable emulsifier), and a weak acid. In such embodiments, the weak acid may have therapeutic benefits in addition to affecting the pH at the wetting layer. For instance, the first solution may include salicylic acid, ascorbic acid, both salicylic acid and ascorbic acid, or a combination of these weak acids with any other weak acid described herein (e.g., citric acid). In particular embodiments, the first solution includes a weak acid (e.g., one or more of salicylic acid, ascorbic acid, and/or citric acid) having a concentration from about 0.5% wt to about 10% wt.

In some embodiments, the activating solution includes only a weak acid in an aqueous solution. It is preferred, however, that the activating solution include one or more emulsifying agents that increase skin or mucosal penetration of the solution. In some embodiments, the activating solution has the same or substantially the same composition as described above for the single solution topical formulation (e.g., comprises a cationic surfactant, one or more emulsifying agents, a microbial biocide, and a weak acid in the concentrations discussed above).

Individual components suitable for use in the two-component system were each described in detail above. For instance, in one embodiment, conditioning solution of the topical formulation system includes a cationic surfactant having a pH greater than about 7; more preferably, greater than about 9 (e.g., potassium cocoate). Suitable conditioning solutions include a cationic surfactant at a concentration in a range from about 10% wt to about 40% wt; preferably, from about 15% wt to about 35% wt, e.g., about 15%, 16%, 17%, 18%, 19%, 20%, 21%, 22%, 23%, 24%, 25%, 26%, 27%, 28%, 29%, 30%, 31%, 32%, 33%, 34%, or 35% wt. The concentration of the biocide, e.g., GME, in the conditioning solution is from about 0.5% wt to about to about 10% wt; preferably, from about 1% wt to about 5% wt, e.g., about 1%, 1.5%, 2%, 2.5%, 3%, 3.5%, 4%, 4.5%, or 5% wt. In other embodiments, the concentration of the biocide is at least about 1% wt; preferably, it is at least about 2% wt.

In a particular embodiment, the conditioning solution includes a pharmaceutically acceptable carrier containing one or more emulsifiers, such as skin permeable emulsifiers (e.g., sorbitan monolaurate (Polysorbate 20), sodium stearoyl lactylate, polyoxyethylene (20) sorbitan monooleate (Polysorbate 80), or any combination thereof). In a non-limiting exemplary embodiment, the emulsifying agents are sorbitan monolaurate (Polysorbate 20) and sodium stearoyl lactylate. In some embodiments, the total concentration of emulsifying agents in the conditioning solution are from about 1% wt to about 50% wt; preferably from about 5% wt to about 40% wt, e.g., about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% wt.

In some embodiments, the pharmaceutically acceptable carrier of the conditioning solution includes one or more nonaqueous oils or gels. For instance, in some embodiments, the pharmaceutically acceptable carrier includes olive oil, vegetable oil, and/or petroleum jelly. In yet other embodiments, the conditioning solution includes thickeners such as synthetic polymers, fatty acids, fatty acid salts and esters, fatty alcohols, modified celluloses or modified mineral materials. In such embodiments, the thickeners can also be employed with liquid carriers to form spreadable pastes, gels, ointments, soaps, and the like, for application directly to the skin of the user.

Suitable activating solution formulations are preferably aqueous solutions and include at least one weak acid or a mixture of one or more weak acids, the weak acid having a pH between about 2 and about 7; preferably, the weak acid will have a pH less than or equal to about 3.5; more preferably, the weak acid will have a pH less than or equal to about 3.0. Non-limiting exemplary weak acids include, but are not limited to, citric acid, lactic acid, malic acid, tartaric acid, salicylic acid, ascorbic acid, and any combination of such weak acids (e.g., a combination of salicylic acid, ascorbic acid, and citric acid). In such embodiments, the concentration of the weak acid in the activating solution is between about 0.2% w/v to about 20% w/v; preferably, between about 0.5% w/v to about 15% w/v, e.g., about 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, or 15.0% w/v. In other embodiments, the concentration of the weak acid in the activating solution is between about 2 g/L to about 200 g/L; preferably, between about 5 g/L to about 150 g/L, e.g., 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, or 150 g/L.

In addition to one or more weak acids, the activating solution may contain one or more emulsifying agents (e.g., skin permeable emulsifiers) to enhance the hyperprotonation and delivery of hydronium to the biomass (e.g., by osmosis or emulsion transport). For instance, GMEs (such as GML) are also known to function as emulsifying agents and can be added to the activating solution. In other embodiments, the emulsifying agents are sorbitan monolaurate (Polysorbate 20), sodium stearoyl lactylate, polyoxyethylene (20) sorbitan monooleate (Polysorbate 80), or any combination thereof. Further, one or more GMEs can be added in addition to sorbitan monolaurate (Polysorbate 20), sodium stearoyl lactylate, polyoxyethylene (20) sorbitan monooleate (Polysorbate 80), or any combination thereof. In such embodiments, the total concentration of emulsifying agents in the activating solution are from about 0.2% to about 10% w/v; preferably, from about 0.5% w/v to about 5% w/v, e.g., about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5% w/v.

In some embodiments, the activating solution is formulated as an aqueous moisturizer gel. In such embodiments, the weak acid is mixed with the aqueous gel, such as hyaluronic acid. In other embodiments, the components of the conditioning formulation are added to or included with cosmetic or makeup products for wear on the skin that is longer than five minutes at a time. In such embodiments, the biocides present in the conditioning formulation will be effectively delivered to the made-up skin. The activating agent can then be incorporated into a makeup remover, which can then be applied to the skin to remove the make up and activate the biocides thereby achieving anti-bacterial, anti-inflammatory, and anti-viral results through standard application and removal of cosmetics, even on a daily or multi-daily basis. Preferably, the activating solution-infused makeup or cosmetic remover would further contain moisturizing ingredients such as hyaluronic acid or other standard moisturizers. A person skilled in the art would be capable of formulating cosmetics, makeup, and makeup remover incorporating the invention disclosed herein achieving a range of efficacious results.

### Methods of Use

The topical formulations provided herein can be applied to the skin or mucosal surface of an individual having a skin condition or disease, or is at risk for developing a skin condition or disease, associated with a microbial biofilm, as microorganisms are the cause of many infectious diseases. Indeed, these microorganisms include pathogenic bacteria that cause diseases such as plague, tuberculosis, and anthrax; protozoa that cause diseases such as malaria, sleeping sickness, dysentery, and toxoplasmosis; and fungi that cause diseases such as ringworm, candidiasis, or histoplasmosis. Other diseases such as influenza, yellow fever or AIDS are caused by pathogenic viruses, which are not usually classified as living organisms, but, for the purposes of this disclosure, are encompassed by the microbial biofilms of the present methods.

Microbial biofilms provide a protective environment in which many of these bacteria, viruses, yeasts, molds, and fungi grow, which can become dormant within these biofilms enabling the reduction of their uptake of antimicrobial agents. These microbial biofilms have therefore been found to be involved in a wide variety of microbial infection in humans and animals, such as urinary tract infections, catheter infections, middle-ear infections, formation of dental plaque, gingivitis, coating contact lenses, and serious and potentially lethal processes such as endocarditis, infections in cystic fibrosis, and infections of permanent indwelling devices such as joint prostheses and heart valves. Microbial biofilms may impair cutaneous wound healing and reduce topical antibacterial efficiency in healing or treating infected skin wounds. Moreover, microbial biofilms are present on the removed tissue of 80% of patients undergoing surgery for chronic sinusitis and can also be formed on the inert surfaces of implanted devices such as catheters, prosthetic cardiac valves, and intrauterine devices. For instance, MRSA is especially troublesome in hospitals, prisons, and nursing homes, where patients with open wounds, invasive devices, and weakened immune systems are at greater risk of nosocomial infection than the general public. MRSA began as a hospital-acquired infection, but has developed limited endemic status and is now sometimes community-acquired. The terms HA-MRSA (healthcare-associated MRSA) and CA-MRSA (community-associated MRSA) reflect this distinction.

Thus, in one embodiment, the topical formulations of the present invention can be used to treat, control, or prevent a variety of eczema conditions, including, but not limited to topic eczema, contact eczema, seborrheic eczema, nummular eczema, neurodermatitis, stasis dermatitis, and dyshidrotic eczema. In some embodiments, the topical formulations described herein are used to treat infected wounds. In other embodiments, the topical formulations described herein are useful for the treatment, control, or prevention of neoplasms, pigmentary disorders, infectious disorders, follicular disorders, hyperkeratotic disorders, inflammatory disorders, vascular disorders, cutaneous cystic disorders, dandruff, seborrheic dermatitis, dry skin, corns, calluses, warts, freckles, acne, wrinkles, cysts, eczema, wounds, insect bites, lupus, varicose veins, tattoos, and/or scars. In addition to humans, the topical formulations of the present invention can be used to treat, control, or prevent skin conditions or skin disease in animals including, but not limited to, pets, livestock, and other animals.

For treating, controlling, or preventing a skin disease or skin condition associated with a microbial biofilm, the topical formulations provided herein are typically applied directly to the affected area (i.e., skin surface or mucosal surface). In one embodiment, the topical formulation is a liquid, cream, gel, or oil, and it is applied to the skin surface or mucosal surface by use of the hands. In other embodiments, it is spread over the affected area via an applicator, such as a wipe, puff, or roller. In yet other embodiments, the topical formulation is a liquid or mist, and it is sprayed onto the skin surface or mucosal surface via a spray bottle. Once applied to the skin surface or mucosal surface, the topical formulations of the present invention can be left on the affected area for a period of about 30 seconds or more, e.g., 30 sec., 40 sec., 50 sec., or more, prior to removing the topical formulation from the affected area (e.g., by rinsing or washing). In other embodiments, the topical formulations are left on the affected area for a period of at least about 1 min., e.g., 1, 2, 3, 4, 5, 6, 7, 8, 9, 10, 11, 12, 13, 14, 15, 16, 17, 18, 19, 20 min., or more.

In another embodiment, a multi-component topical formulation system is used to treat, control, or prevent a skin condition or skin disease associated with a microbial biofilm. For instance, in a preferred embodiment, the topical formulation includes a conditioning solution (i.e., comprising the antimicrobial biocide, cationic surfactant, and emulsifiers) and an activating solution (i.e., comprising the weak acid). In such an embodiment, the conditioning solution can be formulated as a liquid, cream, gel, or oil, and it is applied to the skin surface or mucosal surface by use of the hands or via an applicator, such as a wipe, puff, or roller. In one particular embodiment, the conditioning solution is formulated as a cream and applied to the affected area by use of the hands. Typically, the conditioning solution is left on the affected area for a predetermined amount of time prior to adding the activating solution. In some embodiments, the conditioning solution is left on the affected area for a period of 1 second to about 20 minutes or more. In other embodiments, it is left on the affected area for a period of about 1 second to about 2 minutes. In one particular embodiment, the conditioning solution is left on the affected area for about 10 to about 20 seconds.

Once the conditioning solution has been left on the affected area for the predetermined amount of time, the activating solution is then applied. In some embodiments, the activated solution is formulated as a liquid, cream, gel, oil, or spray, and it is applied to the skin surface or mucosal surface by use of the hands or via an applicator, such as a wipe, puff, roller or spray bottle. In a preferred embodiment, the activating solution is applied to the skin surface or mucosal surface by spraying onto the affected area. Typically, the activating solution is left on the affected area for a predetermined amount of time prior to washing or otherwise removing the topical formulations. In some embodiments, the activating solution is left on the affected area for at least about 1 minute. In other embodiments, it is left on the affected area for at least about 1, 2, 3, 4, 5, 6, 7, 8, 10, 11, 12, 13, 14, 15 minutes or more. In one particular embodiment, the activating solution is left on the affected area for about 1-3 minutes and then washed or wiped off.

In other embodiments, the conditioning solution is incorporated into cosmetic make-up and applied to the skin. When the user removes the make-up he or she can then apply a make-up remover containing the activating solution.

In some embodiments, a topical formulation is applied daily, every other day, biweekly, or weekly; preferably, the topical formulation is applied daily. In other embodiments, a topical formulation is applied 1, 2, 3, 4, 5, 6, 7, 8, 9, 10, or more times per day; preferably, it is applied once per day.

In addition to the topical application to the skin or mucosal surfaces of humans or animals, the compositions and formulations described herein can be used to disinfect or decontaminate hard surfaces or soft surfaces found in household environments, industrial environments, and on food. In another embodiment, the formulations described herein are used to disinfect or decontaminate hard surfaces found in the office or home including, but not limited to, countertops, walls, doors, toilets, shower stalls, bathtubs, bidets, and sinks. In yet another embodiment, the topical formulations are used to disinfect or decontaminate surfaces found in hospitals, medical centers, athletic facilities, gyms, restaurants, hotels, conference centers, and the like. Interior and exterior surfaces of equipment also can be contaminated, including surfaces of equipment used in the food, scientific and medical industries, dental treatment, health care facilities and hospitals.

Moreover, many of the exemplary compositions and methods disclosed herein have the further benefit of being generally regarded as safe (GRAS) by the U.S. FDA for use on food and/or are acceptable under the regulations of the USDA National Organic Production (NOP) and are completely biodegradable.

A person skilled in the art would recognize that the compositions disclosed herein can be made in concentrated form and then diluted to achieve proportions of acids as above. Other compositions are known to provide skin treatments through the use of certain classes of anionic surfactants combined with acidic constituents, such as that described in U.S. Pat. No. 5,143,720, the content of which is incorporated herein by reference in its entirety. A benefit of the invention is that it operates effectively on a broad spectrum basis. It can reliably eradicate both gram-positive and gram-negative microorganisms, as well as combinations of microorganisms where the precise chemical composition is indeterminate.

### Kits and Apparatus

The formulations described above can be packaged for storage, distribution, and use in accordance with any suitable protocol well known to the skilled artisan. For instance, the topical formulation system can be packaged into individual or multi-compartment packs or envelopes for storage and delivery.

Thus, another aspect of the invention comprises kits for use in practice of the present invention. The kits comprise a first container, such as a bottle or tube, that contains the conditioning solution, which includes the cationic surfactant, antimicrobial biocide (e.g., GME), and skin permeable emulsifiers. The conditioning solution may be formulated as a liquid, gel, cream, or ointment. The kits additionally comprise a second container, such as a spray bottle, that contains the activated solution, which includes the weak acid in an aqueous solution. As such, the activating solution may be formulated as a spray. In other embodiments, the conditioning solution is formulated as a gel, cream, or ointment, and the activating solution is formulated as an aqueous gel or cream. In an embodiment, the kit will typically include instructions on how to apply the solutions.

The following examples describe the invention in greater detail. They are intended to illustrate, rather than to limit, the invention.

### EXAMPLES

### Example 1. Exemplary Formulation Compositions

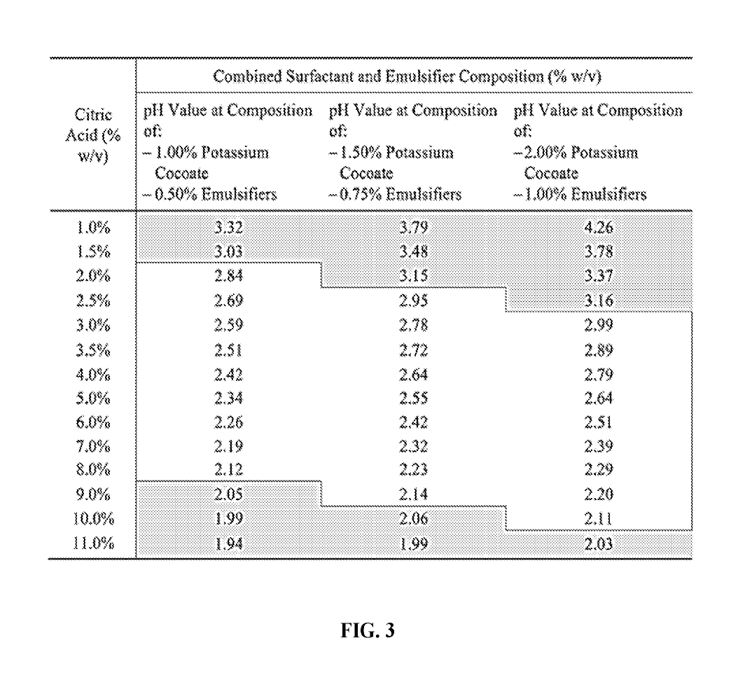

Mixing stable emulsion formulations are well within the purview of the skilled artisan and will not be discussed in detail herein. A non-limiting exemplary single solution topical formulation was produced having the components described in Table 1. Potassium cocoate was chosen as the cationic surfactant, and GML was chosen as the biocide. Furthermore, skin permeable emulsifying agents (i.e., sorbitan monolaurate and sodium stearoyl lactylate) were added. The concentration of citric acid was chosen for this particular formulation based upon how different concentrations of citric acid affect the pH of the potassium cocoate and emulsifier composition (see FIG. 3).

TABLE 1
Exemplary Single Solution Formulation.

| Component | CAS* Registry No. | % w/v | g/L |
|---|---|---|---|
| Water | | 87.00% | 870.00 |
| Potassium Cocoate | 61789-30-8 | 2.00% | 20.00 |
| Sorbitan Monolaurate | 9005-64-5 | 0.80% | 8.00 |
| (Polysorbate 20) | | | |
| Sodium Stearoyl | 25383-99-7 | 0.05% | 0.50 |
| Lactylate | | | |
| GML | 142-18-7 | 0.15% | 1.50 |
| Citric Acid | 77-92-9 | 10.00% | 100.00 |
| Total | | 100% | 1000.00 |

*CAS, Chemical Abstracts Service.

Exemplary topical formulation systems were developed that included a conditioning solution and an activating solution. An embodiment of a conditioning solution is shown in Table 2, and contained much higher concentrations of the surfactant, emulsifiers, and biocide compared to the single solution formulation shown in Table 1. Further, the water content of the conditioning solution was much less than that of the single solution formulation described in Table 1. Three examples of suitable activating solutions were created and shown in Table 2 (Examples A-C). Each were comprised of aqueous solutions containing at least a weak acid. In Examples A and C, emulsifiers were added to increase the delivery and penetration of the acid to the skin. Example C is substantially the same aqueous formulation as described in Table 1 for the single solution formulation and can be added to the skin surface or mucosal surface following application of the conditioning solution.

TABLE 2
Exemplary Topical Formulation Systems.

| | | CAS* | | |
|---|---|---|---|---|
| Component | | Registry No. | wt % | g |
| Conditioning | Water | | 50.00% | 30.00 |
| Solution | Potassium Cocoate | 61789-30-8 | 33.33% | 20.00 |
| | Sorbitan | 9005-64-5 | 13.33% | 8.00 |
| | Monolaurate | | | |
| | (Polysorbate 20) | | | |
| | Sodium Stearoyl | 25383-99-7 | 0.83% | 0.50 |
| | Lactylate | | | |
| | GML | 142-18-7 | 2.50% | 1.50 |
| | Total | | 100.00% | 60.00 |
| | | CAS | | |
| Component | | Registry No. | % w/v | g/L |
| Activating | Water | | 89.15% | 891.50 |
| Solution | Sorbitan | 9005-64-5 | 0.8% | 8.00 |
| (Example A) | Monolaurate | | | |
| | (Polysorbate 20) | | | |
| | Sodium Stearoyl | 25383-99-7 | 0.05% | 0.50 |
| | Lactylate | | | |
| | Citric Acid | 77-92-9 | 10.00% | 100.00 |
| Activating | Water | | 90.00% | 900.00 |
| Solution | Citric Acid | 77-92-9 | 10.00% | 100.00 |
| (Example B) | | | | |
| Activating | Water | | 87.00% | 870.00 |
| Solution | Potassium Cocoate | 61789-30-8 | 2.00% | 20.00 |
| (Example C) | Sorbitan | 9005-64-5 | 0.8% | 8.00 |
| | Monolaurate | | | |
| | (Polysorbate 20) | | | |
| | Sodium Stearoyl | 25383-99-7 | 0.05% | 0.50 |
| | Lactylate | | | |
| | GML | 142-18-7 | 0.15% | 1.50 |
| | Citric Acid | 77-92-9 | 10.00% | 100.00 |

*CAS, Chemical Abstracts Service.

### Example 2. Antimicrobial Performance Testing

An exemplary formulation as described in Table 1 was tested under the conditions described for hospital grade disinfectant according to Schedule 1 of the Therapeutic Goods Order No. 54 and as described in Kelsey and Maurer, Pharm. J. 213:528-530 (1978), the entire content of which is incorporated herein by reference. The formulation was tested neat (with no dilution). The formulation was challenged with bacterial inoculum followed by sampling of this mix at a prescribed time point, rechallenged with the same formulation vial, and sampled again at a later prescribed time point. The sample was cultured in a suitable recovery medium for 48 hr. The organisms used were:

Escherichia coli NCTC 8196;

Pseudomonas aeruginosa NCTC 6749;

Staphylococcus aureus NCTC 4163;

Proteus vulgaris NCTC 4635; and

Listeria monocytogenes A19115.

The formulation was tested with each of these organisms under both ‘clean’ and ‘dirty’ conditions. Clean conditions consisted of resuspension of the test organism in sterile hard water. Dirty conditions consisted of resuspension of the test organism in a sterile yeast suspension (which acted as an organic soil). The formulation passed or failed the assay according to the extent of growth in each of 5 recovery broth tubes at each time point in an assay that was considered valid ie., 10 test vials in total. Validity of the assay depended on the number of organisms/ml in the starting inoculum, which was measured at the time of the assay, and that the expected results were obtained for each of 4 controls. These controls ensured the sterility of the recovery medium, the sterility of the formulation, the growth of the organism and that the formulation was sufficiently inactivated when the sample was added to the recovery medium and therefore allowed the organism to grow if it had not been killed during incubation with the formulation.

For testing, each of the control organisms were required to have been subcultured at least 5, but not more than 14 times (i.e., days in a row). The formulation was required to be tested with each organism under clean and dirty conditions in 3 valid assays carried out over subsequent days.

For E. coli, P. aeruginosa, S. aureus, and P. vulgaris, the contents of an ampoule of freeze-dried culture was incubated overnight at 37° C.+/−1° C. in Wright and Mundy dextrose medium. The incubated culture was inoculated onto nutrient agar slopes in McCartney bottles and stored for up to 3 months at 4° C.+/−1° C. Prior to the test, the culture was subcultured from the agar slope into 10 ml or 15 ml quantities of Wright and Mundy dextrose medium and incubated at 37° C.+/−1° C. for 24+/−2 hours. The subculture was subcultured a second time into fresh medium, using an inoculating loop of about 4 mm in diameter and incubated at 37° C.+/−1° C. for 24+/−2 hours. This step was repeated daily until testing was performed. For the test procedure only those cultures which have been subcultured at least 5, but not more than 14 times, were used.

For L. monocytogenes, a bead from a glycerol stock was inoculated on an HBA plate and incubated overnight at 37° C.+/−1° C. The incubated culture was inoculated onto nutrient agar slopes in McCartney bottles and stored for up to 3 months at 4° C.+/−1° C. Prior to the test, the culture was sub-cultured from the agar slope into 10 ml or 15 ml quantities of BHI medium and incubated at 37° C.+/−1° C. for 24+/−2 hours. The subculture was subcultured a second time into fresh medium, using an inoculating loop of about 4 mm in diameter and incubated at 37° C.+/−1° C. for 24+/−2 hours. This step was repeated daily until testing was performed. For the test procedure only those cultures which have been subcultured at least 5, but not more than 14 times, were used.

Prior to centrifugation, test cultures of P. aeruginosa and S. aureus were filtered through sterile Whatmans No. 4 filter paper. All test cultures were then centrifuged until the cells were compact. Then, the supernatant was removed with a Pasteur pipette, and the test organisms were resuspended in the original volume of liquid (i.e., 10 ml or 15 ml) and shaken for 1 minute with a few sterile glass beads. For the “clean” assay conditions, the test organisms were resuspended in sterile hard water. For the “dirty” assay conditions, the test organisms were resuspended in a mixture of 4 parts yeast suspension to 6 parts sterile hard water.

Immediately before testing, the resuspended inoculums were sampled and enumerated using 10-fold dilutions in quarter-strength Ringer's solution and the pour-plate technique. The number subsequently counted was required to represent not less than 2×10⁸ or more than 2×10⁹ organisms per ml or the test was considered invalid. A tube containing the 10⁻⁷ dilution was used for the controls.

Samples of the formulation was quantitatively diluted to the specified extent, using sterile hard water as diluent. No less than about 10 ml or about 10 g of each sample was used for the first dilution, and no less than 1 ml of any dilution was used to prepare any subsequent dilutions. All dilutions were done in glass containers on the day of testing. The glass containers were twice rinsed in glass-distilled water, and sterilized. Containers were tested at a controlled temperature of 21° C.+/−1° C. either by maintaining the testing environment at this temperature or by use of a water bath.

Next, formulation samples for testing were prepared by adding 3 ml of diluted formulation to a capped glass container and immediately inoculating with 1 ml of test culture and mixing by swirling. At 8 minutes, one drop (0.02 ml+/−0.002 ml) of each formulation sample was subcultured into each of 5 tubes containing recovery broth. At 10 minutes, each formulation sample was inoculated a second time with 1 ml of test culture and mixed by vortexing. At 18 minutes, one drop (0.02 ml+/−0.002 ml) of each formulation sample was subcultured into each of 5 tubes containing recovery broth. All tubes of recovery broth were mixed by vortexing and incubated at 37° C.+/−1° C. for 48+/−2 hours. Next, each tube of recovery broth was examined for growth, and the results were recorded. For each test organism, the test procedure was repeated on each of 2 subsequent days using a fresh formulation sample and a freshly prepared bacterial suspension.

For the recovery broth contamination control, 1 uninoculated tube of recovery broth was incubated at 37° C.+/−1° C. for 48+/−2 hours and examined for growth. If growth occurred, the test was considered invalid due to contamination of the recovery broth. For the formulation contamination control, 0.02 ml of diluted formulation was added to 1 tube of recovery broth and incubated at 37° C.+/−1° C. for 48+/−2 hours and examined for growth. If growth occurred, the test was considered invalid due to contamination of the formulation test sample. To ensure that the test organisms were viable, 1 ml of the 10⁻⁷ microbial dilution obtained above was added to 1 tube of recovery broth and incubated at 37° C.+/−1° C. for 48+/−2 hours and examined for growth. If no growth occurred, the test was considered invalid. To determine the inactivator efficacy, 2 ml of diluted formulation was added to 1 ml of the 10⁻⁷ microbial dilution obtained above and incubated at 37° C.+/−1° C. for 48+/−2 hours and examined for growth. If growth occurred in the organism viability control, but no growth occurred in the formulation/microbial tube, the test was considered invalid due to inadequate inactivation of the formulation. Any invalid test was repeated.

The dilution test passed if there was no apparent growth in at least two out of the five recovery broths in the 8 minute sampling and no apparent growth in at least two of the five recovery broths in the 18 minute sample on all three occasions with all four organisms. As shown in Table 3, the exemplary formulation passed every assay with each test organism under both clean and dirty conditions. For E. coli, P. aeruginosa, S. aureus, and P. vulgaris, no growth was shown in any of the recovery tubes.

TABLE 3
Performance Results.

| | | | | Positive | Positive |
|---|---|---|---|---|---|
| | Assay | Assay | Assay | Cultures | Cultures |
| Organism | Conditions | Number | Validity | at 8 min | at 18 min |
| E. coli | Clean | 1 | Valid | 0 | 0 |
| | | 2 | Valid | 0 | 0 |
| | | 3 | Valid | 0 | 0 |
| | | 4 | Valid | 0 | 0 |
| | Dirty | 1 | Valid | 0 | 0 |
| | | 2 | Invalid | 0 | 0 |
| | | 3 | Valid | 0 | 0 |
| | | 4 | Valid | 0 | 0 |
| P. aeruginosa | Clean | 1 | Valid | 0 | 0 |
| | | 2 | Invalid | 0 | 0 |
| | | 3 | Valid | 0 | 0 |
| | | 4 | Valid | 0 | 0 |
| | Dirty | 1 | Valid | 0 | 0 |
| | | 2 | Valid | 0 | 0 |
| | | 3 | Valid | 0 | 0 |
| S. aureus | Clean | 1 | Valid | 0 | 0 |
| | | 2 | Valid | 0 | 0 |
| | | 3 | Valid | 0 | 0 |
| | Dirty | 1 | Valid | 0 | 0 |
| | | 2 | Valid | 0 | 0 |
| | | 3 | Valid | 0 | 0 |
| P. vulgaris | Clean | 1 | Valid | 0 | 0 |
| | | 2 | Valid | 0 | 0 |
| | | 3 | Valid | 0 | 0 |
| | Dirty | 1 | Invalid | 0 | 0 |
| | | 2 | Valid | 0 | 0 |
| | | 3 | Valid | 0 | 0 |
| | | 4 | Valid | 0 | 0 |
| L. monocytogenes | Clean | 1 | Valid | 0 | 0 |
| | | 2 | Valid | 0 | 0 |
| | | 3 | Valid | 0 | 0 |
| | Dirty | 1 | Valid | 2 | 0 |
| | | 2 | Valid | 1 | 0 |
| | | 3 | Valid | 1 | 0 |

A separate batch of formulation was evaluated in triplicate using the same test protocol described above. Shown in Table 4 are the results for the “clean” assay, whereas the results in Table 5 represent the “dirty” assay.

TABLE 4
Clean Assay Results.

| | | Count | Growth in Recovery Broths | | |
|---|---|---|---|---|---|
| Test | Dilution (v/v) | (Orgs/ml) | Challenge 1 | Challenge 2 | Results |

Escherichia coli NCTC 8196

| | | | | | |
|---|---|---|---|---|---|
| 1 | Neat | 1.4 × 10⁹ | — | — | Pass |
| 2 | Neat | 9.5 × 10⁸ | — | — | Pass |
| 3 | Neat | 4.3 × 10⁸ | — | — | Pass |

Proteus vulgaris NCTC 4635

| | | | | | |
|---|---|---|---|---|---|
| 1 | Neat | 6.5 × 10⁸ | — | — | Pass |
| 2 | Neat | 8.5 × 10⁸ | — | — | Pass |
| 3 | Neat | 5.1 × 10⁸ | — | — | Pass |

Pseudomonas aeruginosa NCTC 6749

| | | | | | |
|---|---|---|---|---|---|
| 1 | Neat | 3.2 × 10⁸ | — | — | Pass |
| 2 | Neat | 5.6 × 10⁸ | — | — | Pass |
| 3 | Neat | 5.8 × 10⁸ | — | — | Pass |

Staphylococcus aureus NCTC 4163

| | | | | | |
|---|---|---|---|---|---|
| 1 | Neat | 2.5 × 10⁸ | — | — | Pass |
| 2 | Neat | 2.5 × 10⁸ | — | — | Pass |
| 3 | Neat | 3.1 × 10⁸ | — | — | Pass |

TABLE 5
Dirty Assay Results.

| | | Count | Growth in Recovery Broths | | |
|---|---|---|---|---|---|
| Test | Dilution (v/v) | (Orgs/ml) | Challenge 1 | Challenge 2 | Results |

Escherichia coli NCTC 8196

| | | | | | |
|---|---|---|---|---|---|
| 1 | Neat | 8.3 × 10⁸ | — | — | Pass |
| 2 | Neat | 8.0 × 10⁸ | — | — | Pass |
| 3 | Neat | 8.8 × 10⁸ | — | — | Pass |

Proteus vulgaris NCTC 4635

| | | | | | |
|---|---|---|---|---|---|
| 1 | Neat | 1.2 × 10⁹ | — | — | Pass |
| 2 | Neat | 2.8 × 10⁸ | — | — | Pass |
| 3 | Neat | 4.7 × 10⁸ | — | — | Pass |

Pseudomonas aeruginosa NCTC 6749

| | | | | | |
|---|---|---|---|---|---|
| 1 | Neat | 1.2 × 10⁹ | — | — | Pass |
| 2 | Neat | 6.5 × 10⁸ | — | — | Pass |
| 3 | Neat | 1.4 × 10⁹ | — | — | Pass |

Staphylococcus aureus NCTC 4163

| | | | | | |
|---|---|---|---|---|---|
| 1 | Neat | 1.3 × 10⁹ | — | — | Pass |
| 2 | Neat | 7.8 × 10⁸ | — | — | Pass |
| 3 | Neat | 4.2 × 10⁸ | — | — | Pass |

The exemplary formulation was further evaluated using the AOAC Hard Surface Carrier Test 991.47,48,49 using undiluted formulation samples. Briefly, the undiluted formulation samples were contacted for 10 minutes with the following test organisms in 5% horse serum:

Pseudomonas aeruginosa ATCC 15442;

Staphylococcus aureus ATCC 6538; and

Salmonella choleraesuis ATCC 10708.

As shown in Table 6, there were only 2 positive carriers for each of the P. aeruginosa and S. aureus samples, whereas the topical formulation eliminated S. choleraesuis in all of the carriers tested.

TABLE 6
Hard Surface Carrier Results.

| | No. | | |
|---|---|---|---|
| | of Carriers | No. of Carriers | No. of Carriers |
| Test Organism | Tested | Negative | Positive |
| Pseudomonas aeruginosa | 60 | 58 | 2 |
| Staphylococcus aureus | 60 | 58 | 2 |
| Salmonella choleraesms | 60 | 60 | 0 |

The exemplary formulation was further evaluated using the BS EN 1276:2009 using 80% v/v diluted formulation samples. Briefly, the formulation samples were contacted for 2, 5, or 10 minutes with Vancomycin resistant Enterococcus faecium or Methicillin resistant Staphylococcus aureus in 0.3% bovine albumin (dirty assay) at 20° C. The results are shown in Table 7.

TABLE 7
Antibiotic Resistant Bacteria Evaluation Results.

| | Initial | | | | | | |
|---|---|---|---|---|---|---|---|
| | Counter per | Final Count per mL | | | Log Reduction | | |
| Organism | mL | 2 min. | 5 min. | 10 min. | 2 min. | 5 min. | 10 min. |
| Vancomycin resistant | 8.1 × 10⁷ | <10 | <10 | <10 | >5.0 | >5.0 | >5.0 |
| Enterococcus faecium | | | | | | | |
| Methicillin resistant | 6.4 × 10⁷ | 1.0 × 10⁵ | 8.0 × 10¹ | <10 | 2.8 | >5.0 | >5.0 |
| Staphylococcus aureus | | | | | | | |

### Example 3. Evaluation of Exemplary Formulations on Microbial Biofilms

A microbial challenge study was performed using microbial biofilms to determine the antimicrobial efficacy of an exemplary formulation with contact times of 30 sec., 1 min., 5 min., and 10 min. against artificially produced biofilms derived from Escherichia coli, Staphylococcus aureus, and Salmonella ssp. Testing was performed in a standard microbiological laboratory employing standard techniques for handling BSL2 microorganisms. Standard PPE and facility notifications per MMDG procedures were followed. Biofilms were developed on borosilicate glass coupons (disks).

A sterile swab of each challenge organism was aseptically taken from stock cultures maintained at 2-8° C. and aseptically transferred to sterile TSA slants. The fresh slants were incubated at 30-35° C. for 18-24 hours. Ten (10) ml of TS saline was pipetted into each slant subsequent to incubation and the growth mechanically dislodged with a sterile cotton-tipped applicator. The suspension was transferred to a sterile 50 ml polypropylene centrifuge tube and washed by centrifugation at 4,000×g for 8-10 min. The supernatant was then decanted and the pellet suspended in 10 ml of saline TS. The suspension was washed a second time and suspended in 10 ml of saline TS. The organism concentration was adjusted to about 10⁸ colony forming units (cfu)/mL based on MMDG historical % T₆₂onm spectrophotometer values.

Disks were wiped with sterile 70% IPA to ensure that no residual oils remained on their surface following handling. The CDC bioreactor was filled to its working volume with 300 mg/L TSB and sterilized in a standard 20-minute liquid steam cycle. The bioreactor was allowed to cool to room temperature. Next, nutritive growth medium (TSB) was prepared at 100 mg/L and sterilized. The bioreactor was acclimated to room temperature. Using sterile tubing, the bioreactor was attached to the source of growth medium. A peristaltic pump was placed between the reactor and the media source to modulate the flow rate. Waste was collected in a separate vessel. Sixteen (16) disks were placed into the reactor representing controls and 12 test surfaces (4 each) for each of 3 antimicrobial challenges. The bioreactor was seeded with one 1 ml of the challenge organism and, operated statically (batch phase) for 24+/−8 hours. The peristaltic pump was turned on following the static operation and the reactor was run in continuous flow mode for an additional 24+/−8 hours at room temperature.

Each disk was removed from the reactor and rinsed gently with sterile TS Saline to remove loosely adhered and planktonic cells and then placed individually into sterile glass beakers containing 10 ml of the test article. The disks were allowed to incubate in the test formulation at ambient temperature for 30 seconds, 1 min., 5 min., and 10 min. Following exposure to the test article, disks were removed from their respective beakers and placed into 10 ml of sterile DEB in a glass test tube to neutralize the test formulation and stop the reaction.

The organisms were removed from the test surfaces and controls through sonication for 20 minutes at room temperature followed by thorough mixing. Serial dilutions of the recovered organisms were performed; 1.0 ml samples of the serial dilutions were plated in duplicate and overpoured with sterile TSA. Plates were incubated under aerobic conditions at 30-35° C. for 3 to 5 days and the recovered organisms quantified.

The log number of microorganisms on the non-treated (no exposure to the test formulation) materials and that of the corresponding materials exposed to the test formulation indicates the reduction in log units.

Log reduction unit=Log A−Log B
Log A=the log number of microorganisms harvested from the non-treated control materials.
Log B=the log number of microorganisms harvested from the corresponding materials exposed to the test formulation.

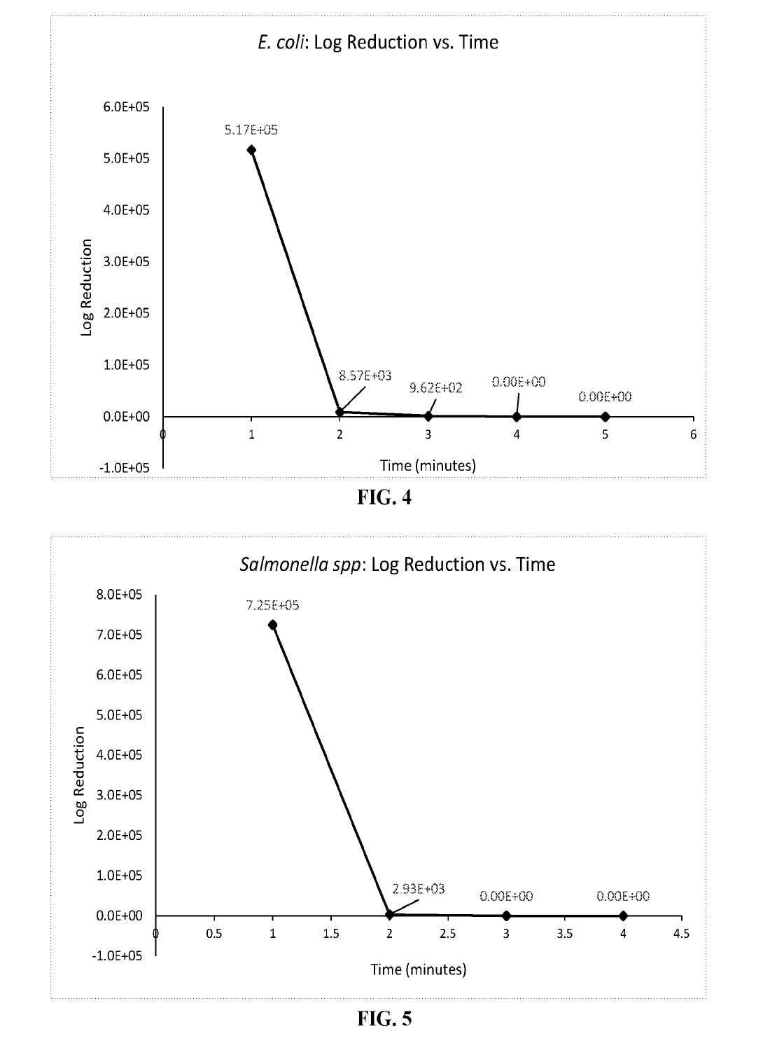

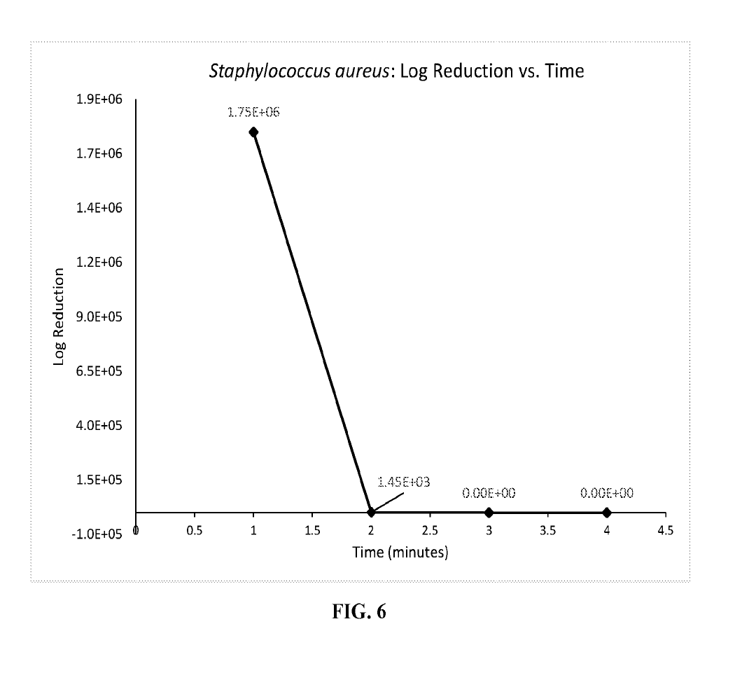

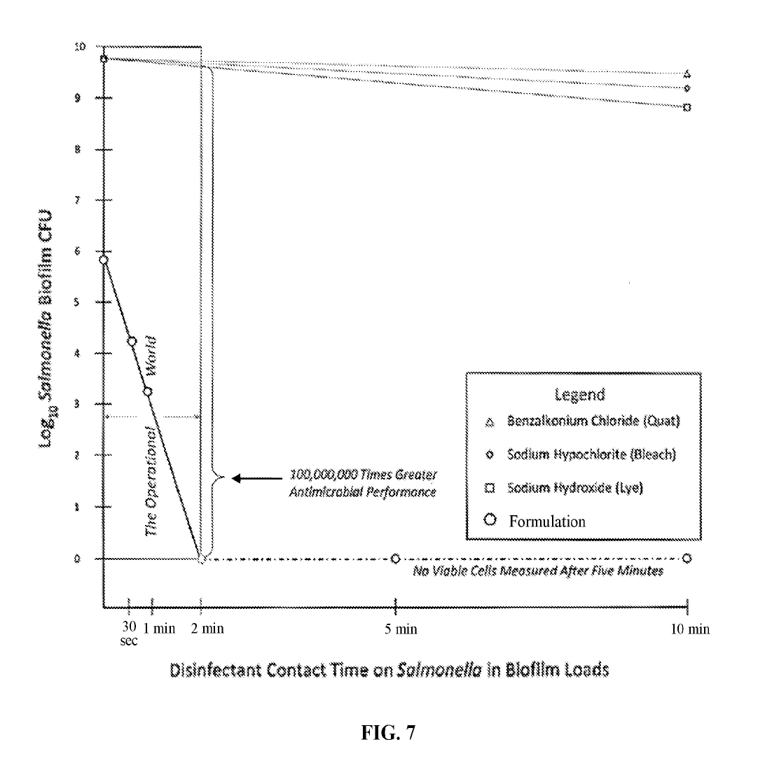

A recovery medium control was performed by first diluting the test formulation 1:10 in DEB and compared to a control sample of 10 ml TSB. Both the DEB and TSB samples were inoculated with about 100 cfu of the challenge organism and 1 ml samples were plated in duplicate. The recovery in the neutralized medium was compared to that of the TSB control. The recovery control results are shown in Table 8, and reveal that the recovery of the microbial challenge for all three organisms was greater than 50%. The results of the microbial biofilm challenge study is shown in Tables 9-12 and FIGS. 4-6. FIG. 7 shows the performance of the formulation as compared to other commercial antibacterial disinfectants.

TABLE 8
Recovery Control Results.
Recovery Medium Control

| | | | | | | | | | % |
|---|---|---|---|---|---|---|---|---|---|
| Organism | Control | CFU | | Ave | Neutralizer | CFU | | Ave | Recovery |
| E. coli | TSB | 122 | 147 | 135 | DEB | 109 | 128 | 119 | 88 |
| Salmonella | TSB | 78 | 86 | 82 | DEB | 66 | 70 | 68 | 83 |
| S. aureus | TSB | 39 | 46 | 43 | DEB | 34 | 50 | 42 | 99 |

TABLE 9
E. coli Challenge Results.

| | | | CFU recovered | | CFU recovered | | CFU recovered | | | Average × |
|---|---|---|---|---|---|---|---|---|---|---|
| Sample | | Dilution | #1 | | #2 | | #3 | | Average | Dilution |
| Control | | 1 × 10⁴ | 51 | 46 | 33 | 69 | 48 | 63 | 52 | 5.17 × 10⁵ |
| 30 | sec. | 1 × 10² | 79 | 77 | 103 | 94 | 88 | 73 | 86 | 8.57 × 10³ |
| 1 | min. | 1 × 10¹ | 99 | 81 | 106 | 101 | 97 | 93 | 93 | 9.62 × 10² |
| 5 | min. | 1 × 10⁰ | 0 | 0 | 0 | 0 | 0 | 0 | 0 | — |
| 10 | min. | 1 × 10⁰ | 0 | 0 | 0 | 0 | 0 | 0 | 0 | — |

TABLE 10
Salmonella spp. Challenge Results.

| | | | CFU recovered | | CFU recovered | | CFU recovered | | | Average × |
|---|---|---|---|---|---|---|---|---|---|---|
| Sample | | Dilution | #1 | | #2 | | #3 | | Average | Dilution |
| Control | | 1 × 10⁴ | 51 | 39 | 106 | 101 | 60 | 78 | 73 | 7.25 × 10⁵ |
| 1 | min. | 1 × 10¹ | 269 | 301 | 312 | 319 | 285 | 270 | 293 | 2.93 × 10² |
| 5 | min. | 1 × 10⁰ | 0 | 0 | 0 | 0 | 0 | 0 | 0 | — |
| 10 | min. | 1 × 10⁰ | 0 | 0 | 0 | 0 | 0 | 0 | 0 | — |

TABLE 11
S. aureus Challenge Results.

| | | | CFU recovered | | CFU recovered | | CFU recovered | | | Average × |
|---|---|---|---|---|---|---|---|---|---|---|
| Sample | | Dilution | #1 | | #2 | | #3 | | Average | Dilution |
| Control | | 1 × 10⁴ | 194 | 171 | 156 | 183 | 180 | 166 | 175 | 1.75 × 10⁶ |
| 1 | min. | 1 × 10¹ | 144 | 157 | 130 | 139 | 155 | 142 | 145 | 1.45 × 10³ |
| 5 | min. | 1 × 10⁰ | 0 | 0 | 0 | 0 | 0 | 0 | 0 | — |
| 10 | min. | 1 × 10⁰ | 0 | 0 | 0 | 0 | 0 | 0 | 0 | — |

TABLE 12
Antimicrobial Properties vs. Time.

| Time | E. coli (CFU) | Salmonella (CFU) | Staph. (CFU) |
|---|---|---|---|
| 0 | 517,000 | 725,000 | 1,750,000 |
| 30 sec. | 8,570 | 11,900 | 28,600 |
| 1 min. | 962 | 2,930 | 1,450 |
| 5 min. | 0 | 0 | 0 |

### Example 4. Evaluation of the Multi-Component Topical Formulation System

An exemplary two-solution topical formulation system was prepared as described in Table 2. The two-solution formulation system was evaluated to determine its efficacy in treating skin conditions and skin diseases in individuals.

In one study, 15 people were identified with eczema and dermatitis of varying severity. For each individual, the two-solution topical formulation system was administered to the affected area. First, the conditioning solution was applied and left on the affected area for about 10 seconds. Next, the activating solution was applied and left on the affected area for about 2 minutes. The solutions were washed off. The individuals were instructed to apply the two-solution topical formulation system once a day. It was also recommended to the individuals to apply a moisturizer.

In 14 out of 15 cases, there were significant positive results. On average, the itching associated with eczema stopped within 2 minutes of application, and the skin was significantly improved within 3 to 5 days of treatment. One individual had a slightly adverse reaction to the topical formulation which resulted in irritation due to an underlying allergy to citric acid.

One individual had been suffering from dermatitis for about 15 years. After application of the topical formulation system 5 to 6 times over approximately 2 weeks, the condition had completely cleared. Another 80-year-old male had suffered from eczema for 50 years. After the first application of the topical formulation system, the itching stopped. After 4 applications, his skin became significantly clearer and his wounds healed. A 14-year-old boy with eczema behind the ear and on his head saw clearing of the skin after 3 applications.

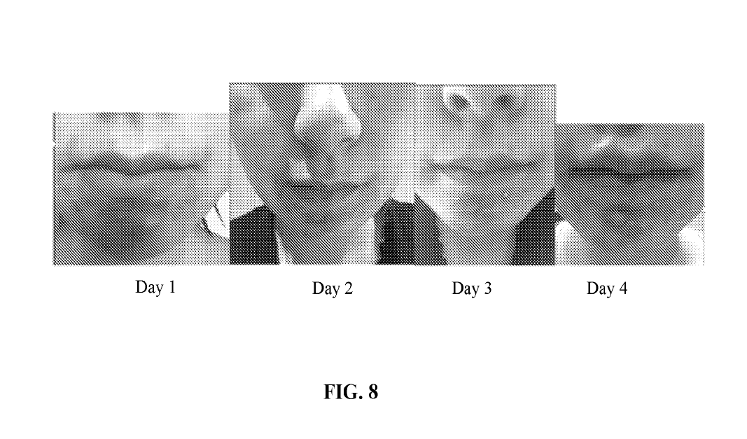

In another study, about two dozen people showed that the topical formulation system was effective in treating pimples and acne. For example, 4 people with cystic acne experienced reduced inflammation in a matter of days following treatment. FIG. 8 illustrates the results seen in one individual suffering from cystic acne over a four day period of daily treatments with the topical formulation system.

### REFERENCES

Barger et al., Cleaning and disinfecting compositions with electrolytic disinfecting booster, U.S. Pat. No. 6,255,270 (Jul. 3, 2001)
Bueg-Deeb, et al., Methods and compositions for cleaning and disinfecting surfaces, U.S. Pub. No. 2014/0275267 (Sep. 18, 2014)
W. G. Characklis, Microbial Biofouling Control in Biofilms, Characklis and Marshall, eds., Wiley & Sons, 1990, J. W. Costerton, Ann Rev. Microb. 49:7110-7145, 1995
Donelli, G. ed., Biofilm-based Healthcare-associated Infections, Volume 1 (Springer 2015)
Donelli, G. ed., Biofilm-based Healthcare-associated Infections, Volume 2 (Springer 2015),
Hidalgo, et al., Functional Tomographic Fluorescence Imaging of pH Microenvironments in Microbial Biofilms by Use of Silica Nanoparticle Sensors, Appl Environ Microbiol. 2009 December; 75(23): 7426-7435
James et al., Wound Rep Reg. 16: 37-44, 2008
McCutcheon's, Detergents and Emulsifiers, North American Edition, 1991
Nusbaum et al., Biofilms in Dermatology, Skin Therapy Lett. (2012 July; 17(7))
Ruzin, et al., Equivalence of Lauric Acid and Glycerol Monolaurate as Inhibitors of Signal Transmission in Styaphyloccus aures, Journal of Bacteriology (May 2000)
Schlievert, et al., Glycerol Monolaurate Antibacterial Activity in Broth and Biofilm Cultures, PloS One (2012)
Schlievert, Compositions for topical treatment of microbial infections, U.S. Pub. No. 2013/0281532 (Oct. 24, 2013)
Silvester, Amine-acid thickening compositions, EP 0253676 (1988)
Singh and Barbul, Wound Rep Reg. 16: 1, 2008
Urban, Hard surface cleaning compositions and method of removing stains, U.S. Pat. No. 6,936,579 (Aug. 30, 2005)
Whitley et al., Biofilm remover, U.S. Pat. No. 8,012,461 (Sep. 6, 2011)
Physicochemical characterization and evaluation of a microemulsion system for antimicrobial activity of glycerol monolaurate, doi:10.1016/j.ijpharm.2006.05.019
Compositions for topical treatment of microbial infections, WO 2013159029
Inhibition of biofilm formation by 1,2,3,4,6-penta-o-galloyl-d-glucopyranose, U.S. Pub. No. 2012/0321566
COMPOSITIONS FOR TREATING BIOFILM, U.S. Pat. No. 6,830,745
A composition for use on skin and wound, WO 2010070292
CONTROL OF BIOFILM FORMATION, U.S. Pub. No. 2014/0205586
INHIBITING BACTERIAL INFECTION AND BIOFILM FORMATION, EP 2 509 622
Antimicrobial-antibiofilm compositions and methods of use thereof, WO 2014134701
Hess et al., Antibacterial Synergy of Glycerol Monolaurate and Aminoglycosides in Staphylococcus aureus Biofilms, Antimicrob Agents Chemother. 2014 November; 58(11):6970-3.
Hess et al. The Natural Surfactant Glycerol Monolaurate Significantly Reduces Development of Staphylococcus aureus and Enterococcus faecalis Biofilm, Surgical Infections. October 2015, 16(5): 538-542. doi:10.1089/sur.2014.162.
Allen et al., The presence and impact of biofilm-producing staphylococci in atopic dermatitis, JAMA Dermatol. 2014 March; 150(3):260-5. doi: 10.1001/jamadermatol.2013.8627
Lieberman et al., A Review of Monolaurin and Lauric Acid: Natural Virucidal and Bactericidal Agents, Alternative and Complementary Therapies. December 2006, 12(6): 310-314. doi:10.1089/act.2006.12.310.
Stoodley, P., D. de Beer, and H. M. Lappin-Scott, Influence of Electric Fields and pH on Biofilm Structure as Related to the Bioelectric Effect, Antimicrob. Agents Chemother., 41(9):189 (1997).
69 Federal Register 34,937, C8, C10, and C12 Straight-Chain Fatty Acid Monoesters of Glycerol and Propylene Glycol; Exemption from the Requirement of a Tolerance (Jun. 23, 2004)

I claim:

1. A topical formulation system for treatment, control or prevention of a skin condition or skin disease associated with a microbial biofilm, the topical formulation system comprising a set of reaction components comprising:
(a) a first reaction component comprising:
(i) a cationic surfactant in an amount from about 10% wt to about 40% wt of the first reaction component;
(ii) a pharmaceutically acceptable carrier comprising one or more emulsifying agents, wherein a total amount of emulsifying agents in the first reaction component is from about 5% wt to about 40% wt; and
(iii) a dermatologically acceptable biocide in an amount of at least about 1% wt of the first reaction component;
(b) a second reaction component comprising an aqueous solution comprising one or more weak acids having a total amount of weak acids in a range from about 0.5% w/v to about 15% w/v of the second reaction component, wherein:
(i) the one or more weak acids have a pH in a range from about 2 to about 6;
(ii) the one or more weak acids comprises a first titration point pH; and
(iii) the cationic surfactant of the first component has a pH of at least about 2 units greater than the first titration point pH of the one or more weak acids;
wherein combining the reaction components in (a) and (b) on a mucosal surface or skin surface comprising a microbial biofilm produces a wetting layer, wherein the wetting layer increases protonation of water to produce hydronium, and wherein the wetting layer increases delivery of the hydronium and the dermatologically acceptable biocide to the microbial biofilm thereby disrupting the microbial biofilm.

2. The topical formulation system of claim 1, wherein the second reaction component further comprises one or more emulsifying agents, wherein a total amount of emulsifying agents in the second reaction component is from about 0.2% w/v to about 10% w/v.

3. The topical formulation system of claim 2, wherein the one or more emulsifying agents in the second reaction component are skin permeable.

4. The topical formulation system of claim 3, wherein the one or more emulsifying agents in the second reaction component are selected from the group consisting of a glycerol monoester, sorbitan monolaurate, sodium stearoyl lactylate, polyoxyethylene (20) sorbitan monooleate, and any combination thereof.

5. The topical formulation system of claim 1, wherein the cationic surfactant is a fatty acid salt or a saponified organic acid, and wherein the pH of the one or more weak acids is less than about 3.5.

6. The topical formulation system of claim 5, wherein the cationic surfactant is potassium cocoate.

7. The topical formulation system of claim 5, wherein the one or more weak acids are selected from the group consisting of ascorbic acid, citric acid, salicylic acid, lactic acid, malic acid, tartaric acid, and any combination thereof.

8. The topical formulation system of claim 1, wherein the pharmaceutically acceptable carrier further comprises one or more nonaqueous oils or gels.

9. The topical formulation system of claim 8, wherein the one or more nonaqueous oils or gels are selected from the group consisting of olive oil, vegetable oil, petroleum jelly, and any combination thereof.

10. The topical formulation system of claim 1, wherein the one or more emulsifying agents in the first reaction component are skin permeable.

11. The topical formulation system of claim 10, wherein the one or more emulsifying agents in the first reaction component are selected from the group consisting of sorbitan monolaurate, sodium stearoyl lactylate, polyoxyethylene (20) sorbitan monooleate, and any combination thereof.

12. The topical formulation system of claim 1, wherein the first reaction component is formulated for application as liquid, cream or gel.

13. The topical formulation system of claim 1, wherein the second reaction component is formulated for application as a spray or an aqueous gel.

14. The topical formulation system of claim 13, wherein the second reaction component is formulation for an aqueous gel comprising hyaluronic acid.

15. The topical formulation system of claim 1, wherein the first reaction component further comprises one or more weak acids selected from the group consisting of salicylic acid, ascorbic acid, citric acid, and any combination thereof, and wherein the total concentration of weak acids in the first reaction component is a least about 0.5% wt.

16. The topical formulation system of claim 1, wherein combining the first reaction component and the second reaction component on a mucosal surface or skin surface comprising a microbial biofilm produces a stable emulsified mixture in accordance with the hydrophilic-lipophilic balance system.

17. The topical formulation system of any one of the preceding claims, wherein the dermatologically acceptable biocide is a glycol monoester of the formula:
R₁OCH₂(OR₂)CH₂OR₃
wherein R₁, R₂ and R₃ are individually H or a C6 to C22 acyl group.

18. The topical formulation system of claim 17, wherein the glycol monoester is selected from the group consisting of glycerol monocaprylate, glycerol monocaprate, glycerol monolaurate, glycerol monomyristate, and any combination thereof.

19. The topical formulation system of claim 18, wherein the glycol monoester is glycerol monolaurate at a concentration of greater than about 2% wt, and wherein the cationic surfactant in the first reaction component is in an amount from about 15% wt to about 35% wt.

20. A method for treating, controlling or preventing a skin disease or skin condition associated with a microbial biofilm in an individual, the method comprising:
(a) providing an individual having a mucosal surface or skin surface comprising a microbial biofilm;
(b) applying a conditioning solution to the mucosal surface or skin surface of the individual, wherein the conditioning solution comprises:
(1) a cationic surfactant in an amount from about 15% wt to about 35% wt;
(2) a pharmaceutically acceptable carrier comprising one or more skin permeable emulsifying agents, wherein a total amount of skin permeable emulsifying agents is from about 5% wt to about 40% wt; and
(3) a dermatologically acceptable biocide in an amount of at least about 2% wt, wherein the biocide is a glycol monoester of the formula:
R₁OCH₂(OR₂)CH₂OR₃
wherein R₁, R₂ and R₃ are individually H or a C6 to C22 acyl group; and
(c) applying an activating solution to the mucosal surface or skin surface of the individual, wherein the activating solution comprises an aqueous solution comprising one or more weak acids having a total amount of weak acids ranging from about 0.5% w/v to about 15% w/v, and wherein:
(1) the one or more weak acids have a pH in a range from about 2 to about 6;
(2) the one or more weak acids comprises a first titration point pH; and
(3) the cationic surfactant of the first component has a pH of at least about 2 units greater than the first titration point pH of the one or more weak acids;
wherein a combination of the conditioning solution and the activating solution at the mucosal surface or skin surface of the individual produces a wetting layer, wherein the wetting layer increases protonation of water to produce hydronium, and wherein the wetting layer increases delivery of the hydronium and the dermatologically acceptable biocide to the microbial biofilm thereby disrupting the microbial biofilm and treating, controlling or preventing the skin disease or skin condition associated with the microbial biofilm in the individual.

21. The method of claim 20, wherein the activating solution further comprises one or more skin permeable emulsifying agents having a total amount ranging from about 0.2% w/v to about 10% w/v, and wherein the one or more skin permeable emulsifying agents in the activating solution are selected from the group consisting of a glycerol monoester, sorbitan monolaurate, sodium stearoyl lactylate, polyoxyethylene (20) sorbitan monooleate, and any combination thereof.

22. The method of claim 20, wherein the cationic surfactant is a fatty acid salt or a saponified organic acid, and wherein the pH of the one or more weak acids is less than about 3.5.

23. The method of claim 22, wherein the cationic surfactant is potassium cocoate.

24. The method of claim 22, wherein the one or more weak acids are selected from the group consisting of ascorbic acid, citric acid, salicylic acid, lactic acid, malic acid, tartaric acid, and any combination thereof.

25. The method of claim 20, wherein the pharmaceutically acceptable carrier further comprises one or more nonaqueous oils or gels.

26. The method of claim 25, wherein the one or more nonaqueous oils or gels are selected from the group consisting of olive oil, vegetable oil, petroleum jelly, and any combination thereof.

27. The method of claim 20, wherein the one or more skin permeable emulsifying agents in the conditioning solution are selected from the group consisting of sorbitan monolaurate, sodium stearoyl lactylate, polyoxyethylene (20) sorbitan monooleate, and any combination thereof.

28. The method of claim 20, wherein the conditioning solution is formulated for application as liquid, cream, or gel, and wherein the activating solution is formulated for application as a spray.

29. The method of claim 20, wherein the conditioning solution is into a cosmetic product, and wherein the activating solution is incorporated into a cosmetic product remover.

30. The method of claim 20, wherein the combination of the conditioning solution and the activating solution at the mucosal surface or skin surface of the individual produces a stable emulsified mixture in accordance with the hydrophilic-lipophilic balance system.

31. The method of claim 20, wherein the skin condition or skin disease is selected from the group consisting of atopic dermatitis, eczema, acne vulgaris, warts, wound infection, fungal skin disease, and viral skin disease.

32. The method of claim 20, wherein the individual is a human or animal.

33. The method of any one of claims 20-32, wherein the activating solution is applied about 10 seconds to about 30 seconds after application of the conditioning solution.

34. The method of any one of claims 20-32, wherein the glycol monoester is selected from the group consisting of glycerol monocaprylate, glycerol monocaprate, glycerol monolaurate, glycerol monomyristate, and any combination thereof.

35. A topical formulation for treatment, control or prevention of a skin condition or skin disease associated with a microbial biofilm, the topical formulation comprising:
(a) a cationic surfactant in an amount from about 1% w/v to about 5% w/v;
(b) one or more skin permeable emulsifying agents, wherein a total amount of skin permeable emulsifying agents is from about 0.5% w/v to about 5% w/v;
(c) a dermatologically acceptable biocide in an amount of at least about 0.1% w/v, wherein the dermatologically acceptable biocide is a glycol monoester of the formula:
R₁OCH₂(OR₂)CH₂OR₃
wherein R₁, R₂ and R₃ are individually H or a C6 to C22 acyl group; and
(d) at least one weak acid in an amount from about 0.5% w/v to about 15% w/v, wherein:
(1) the at least one weak acid has a pH in a range from about 2 to about 6;
(2) the at least one weak acid comprises a first titration point pH; and
(3) the cationic surfactant has a pH of at least about 2 greater than the first titration point pH of the at least one weak acid; and
wherein a wetting layer is formed upon application of the topical formulation on a mucosal surface or skin surface comprising a microbial biofilm, wherein the wetting layer increases protonation of water to produce hydronium, and wherein the wetting layer increases delivery of the hydronium and the dermatologically acceptable biocide to the microbial biofilm thereby disrupting the microbial biofilm.

36. The topical formulation of claim 35, wherein the cationic surfactant is a fatty acid salt or a saponified organic acid, and wherein the pH of the at least one weak acid is less than about 3.5.

37. The topical formulation of claim 36, wherein the cationic surfactant is potassium cocoate.

38. The topical formulation of claim 36, wherein the at least one weak acid is selected from the group consisting of ascorbic acid, salicylic acid, citric acid, lactic acid, malic acid, tartaric acid, and any combination thereof.

39. The topical formulation of claim 35, further comprising one or more nonaqueous oils or gels.

40. The topical formulation of claim 39, wherein the one or more nonaqueous oils or gels are selected from the group consisting of olive oil, vegetable oil, petroleum jelly, and any combination thereof.

41. The topical formulation of claim 35, wherein the one or more skin permeable emulsifying agents are selected from the group consisting of sorbitan monolaurate, sodium stearoyl lactylate, polyoxyethylene (20) sorbitan monooleate, and any combination thereof.

42. The topical formulation of claim 35, wherein the topical formulation is formulated for application as liquid, cream, gel or spray.

43. The topical formulation of claim 35, wherein the formulation produces a stable emulsified mixture in accordance with the hydrophilic-lipophilic balance system.

44. The topical formulation of any one of claims 35-43, wherein the glycol monoester is selected from the group consisting of glycerol monocaprylate, glycerol monocaprate, glycerol monolaurate, glycerol monomyristate, and any combination thereof.

45. The topical formulation of claim 44, wherein the glycol monoester is glycerol monolaurate at a concentration of greater than about 500 μg/ml.

46. A method for treating, controlling or preventing a skin disease or skin condition associated with a microbial biofilm in an individual, the method comprising:
(a) providing an individual having a mucosal surface or skin surface comprising the microbial biofilm; and
(b) applying to the mucosal surface or skin surface of the individual the topical formulation of any one of claims 35-45, wherein application of the topical formulation disrupts the microbial biofilm thereby treating, controlling or preventing the skin disease or skin condition associated with the microbial biofilm in the individual.

47. The method of claim 46, wherein the skin condition or skin disease is selected from the group consisting of atopic dermatitis, eczema, acne vulgaris, warts, wound infection, fungal skin disease, and viral skin disease.

48. The method of claim 47, wherein the individual is a human or animal.

49. A multi-layered composition for enhanced delivery of hydronium, the composition comprising:
(a) a surface layer on which is disposed a microbial biofilm;
(b) a wetting layer disposed on the surface layer, the wetting layer comprising a cationic surfactant, one or more emulsifying agents, and a biocide having the formula
R₁OCH₂(OR₂)CH₂OR₃
wherein R₁, R₂ and R₃ are individually H or a C6 to C22 acyl group; and
(c) an emulsion layer disposed on the wetting layer; the emulsion layer comprising water and one or more weak acids;
wherein the wetting layer and emulsion layer comprises a total weight, and wherein:
(i) the cationic surfactant is in an amount from about 10% wt to about 40% wt of the total weight;
(ii) the one or more emulsifying agents are in an amount from about 5% wt to about 40% wt of the total weight;
(iii) the biocide is in an amount of at least about 1% wt of the total weight;
(iv) the one or more weak acids are an amount from about 0.5% wt to about 15% wt of the total weight;
(vi) the one or more weak acids comprise a first titration point and have a pH in a range from about 2 to about 6; and
(vii) the cationic surfactant has a pH of at least about 2 units greater than the first titration point pH of the one or more weak acids;
wherein the wetting layer increases protonation of water to produce hydronium, and wherein the wetting layer increases delivery of the hydronium and the biocide to the microbial biofilm thereby disrupting the microbial biofilm.

50. The composition of claim 49, wherein the cationic surfactant is a fatty acid salt or a saponified organic acid and wherein the pH of the at least one weak acid is less than about 3.5.

51. The composition of claim 50, wherein the cationic surfactant is potassium cocoate, and wherein the one or more weak acids are selected from the group consisting of ascorbic acid, salicylic acid, citric acid, lactic acid, malic acid, tartaric acid, and any combination thereof.

52. The composition of claim 49, wherein the one or more emulsifying agents are selected from the group consisting of sorbitan monolaurate, sodium stearoyl lactylate, polyoxyethylene (20) sorbitan monooleate, and any combination thereof.

53. The composition of claim 49, wherein the surface is a skin surface or mucosal surface.

54. The composition of of any one of claims 49-53, wherein the glycol monoester is selected from the group consisting of glycerol monocaprylate, glycerol monocaprate, glycerol monolaurate, glycerol monomyristate, and any combination thereof.
